(19) United States
(12) Patent Application Publication
(10) Pub. No.: US 2023/0295237 A1
(43) Pub. Date: Sep. 21, 2023

(54) COMPOSITION AND METHOD

(71) Applicant: Cambridge Enterprise Limited, Cambridge Cambridgeshire (GB)

(72) Inventors: Robin J M Franklin, Cambridge Cambridgeshire (GB); Bjorn Neumann, Cambridge Cambridgeshire (GB); Michael Segel, Cambridge Cambridgeshire (GB); Adam Young, Cambridge Cambridgeshire (GB)

(21) Appl. No.: 18/007,378

(22) PCT Filed: Jul. 30, 2021
(86) PCT No.: PCT/GB2021/051985

§ 371 Date: Jan. 30, 2023

(30) Foreign Application Priority Data
Jul. 30, 2020 (GB) 2011871.7

(51) Int. Cl. C07K 14/005; C12N 15/86; A61P 25/00

(52) U.S. Cl. CPC C07K 14/005; A61P 25/00; C12N 15/86; A61K 38/00; C12N 2750/14122; C12N 2750/14143; C12N 2750/14171

(57) ABSTRACT

The present invention relates to adeno-associated virus (AAV) capsid proteins that have been modified to insert an amino acid sequence and/or methods of targeting microglia or brain macrophages using the AAV capsid proteins of the invention.

### FIELD OF THE INVENTION

The present invention relates to adeno-associated virus (AAV) capsid proteins that have been modified to insert an amino acid sequence and/or methods of targeting microglia or brain macrophages using the AAV capsid proteins of the invention.

### BACKGROUND OF THE INVENTION

Microglia are tissue resident macrophages of the central nervous system (CNS) and play critical roles in CNS immune defence, development and homeostasis (Schafer and Stevens 2015; Li and Barres 2018; Salter and Stevens 2017; Wolf et al., 2017; Prinz et al., 2019). These highly dynamic cells continually react to their environment (Gosselin et al. 2017). Genetic studies also strongly implicate microglial dysfunction in neurodegeneration, neuroinflammation, as well as neuropsychiatric and neurodevelopmental conditions (Guerreiro et al. 2013; Jonsson et al. 2013; Tansey, Cameron, and Hill 2018; Gjoneska et al. 2015; Young et al., 2019). Functionally, microglia appear to lose capacity with ageing, particularly in the context of neurodegenerative and neuroinflammatory conditions. A specific example of this, is in the context of chronic demyelinating disease such as multiple sclerosis (MS) whereby the reduced ability of microglia both to populate a site of injury and undertake phagocytosis of debris decreases with ageing (Kotter et al., 2006; Natrajan et al., 2015; Ruckh et al., 2012). Moreover, human studies further indicate that microglia may be the damaging elements in MS, whereby the abundance of activated microglial cells is an early event preceding demyelination and lesion formation, which correlates with clinical disability.

In the context of neurodegeneration, specific changes in microglia functions are associated with Parkinson’s and Alzheimer’s disease, whereby proinflammatory microglia have been shown to associate with Aβ plaques. Furthermore, reduced phagocytic activity of microglia is characteristic of Amyotophic Lateral Sclerosis (Wolf et al. 2017). With regard to neuropsychiatric disorders, changes in microglia density and morphology in distinct brain regions has been demonstrated in Autism Spectrum Disorder patients, whilst abnormalities in microglial activation accompanied by alterations in neuroinflammation are observed in patients with schizophrenia (Leza et al. 2015).

Microglia are attractive targets for therapy in the CNS primarily because they are abundant and can self-renew. At present, there has been limited intervention with non-specific treatments such as inhibitors of CSF1R, which have been used in animal models of Alzheimer’ Disease (Spangenberg et al., 2019). However, with recent evidence that microglia exist in different functional states in both animal models and humans (Young et al., 2019; Olah et al. 2018; Keren-Shaul et al. 2017; Hammond et al. 2019; Masuda et al. 2019; Mrdjen et al. 2018; Mathys et al. 2017), a more attractive proposition would be to selectively and precisely functionally gene edit relevant sub-populations of cells.

The creation of the PHP.B plasmid allows infection of the CNS with high efficiency via the systemic circulation (Deverman et al., 2016). More recently, PHP.eB has been used to perform gene editing of stem cells in the CNS (Segel et al., 2019). Whilst PHP.eB has been shown to be effective at transducing neurons, it has not proven possible to achieve in vivo infection of microglia with PHP.eB (Deverman et al., 2016; Kumar et al., 2020) .

There is thus a need for improved compositions and methods for targeting microglia in vivo. The present invention addresses this need by providing novel AAV capsid proteins and AAV particles that are also capable of infecting microglia and brain resident macrophages, which include CNS-infiltrating macrophages derived from recruited monocytes.

### SUMMARY OF THE INVENTION

The present invention is based on the creation of novel AAV capsid proteins that can be introduced into AAV viral particles. AAVs having the modified capsid proteins are capable of crossing the blood brain barrier to efficiently target cells throughout the CNS, including microglia and brain macrophages, which include CNS-infiltrating macrophages derived from recruited monocytes.

These novel AAV capsid proteins have been modified to insert an amino acid sequence in the AAV capsid binding arm. The novel AAV capsid proteins of the invention can be incorporated into viral particles that are particularly effective at targeting microglial cells. Thus, AAVs comprising the novel AAV capsid proteins of the invention are highly advantageous compared to previously described AAVs as they demonstrate efficient transduction in cells throughout the CNS.

Accordingly, the invention provides:

An adeno-associated virus (AAV) capsid protein, wherein the AAV capsid protein has been modified to insert an amino acid sequence having:
(a) the amino acid sequence of SEQ ID NO: 1, or a variant thereof having a single amino acid substitution;
(b) the amino acid sequence of SEQ ID NO: 2, or a variant thereof having a single amino acid substitution;
(c) the amino acid sequence of SEQ ID NO: 3, or a variant thereof having a single amino acid substitution;
(d) the amino acid sequence of SEQ ID NO: 4, or a variant thereof having a single amino acid substitution;
(e) the amino acid sequence of SEQ ID NO: 5, or a variant thereof having a single amino acid substitution;
(f) the amino acid sequence of SEQ ID NO: 6, or a variant thereof having a single amino acid substitution;
(g) the amino acid sequence of SEQ ID NO: 7, or a variant thereof having a single amino acid substitution;
(h) the amino acid sequence of SEQ ID NO: 8, or a variant thereof having a single amino acid substitution; or
(i) the amino acid sequence of SEQ ID NO: 9.

The invention also provides:

A nucleic acid encoding the AAV capsid protein according to the invention; a recombinant DNA comprising the nucleic acid according to the invention; a host cell comprising the nucleic acid or the recombinant DNA according to the invention; a viral particle comprising the AAV capsid protein according to the invention; a host cell that produces the viral particle of the invention; a non-human transgenic animal comprising the viral particle according to the invention; or a pharmaceutical composition comprising the AAV capsid protein, the nucleic acid or the viral particle according to the invention; and one or more pharmaceutically acceptable excipients.

The invention also provides:

A method of targeting microglia or brain macrophages using an AAV capsid protein according to the invention; the method comprising introducing a recombinant AAV vector into a mammal; the recombinant AAV vector encoding for a gene of interest, a gene editing construct, an antibody or antigen-binding fragment, or a gene silencing construct, which is encapsidated into a capsid protein according to the invention.

### BRIEF DESCRIPTION OF THE FIGURES

FIG. 1: Illustration of vector design to create infection random library. (A) Schematic of PHP.eB AAV containing random library insertion. (B) Transfer vector used to create novel virus library. PHP.eB capsid sequence is split at amino acid position 588 and PHP.eB binding arm is replaced by a random library of 21 base pairs which have the capability of generating 1.9448e⁹ different viruses. The plasmid encodes further for a GFP fluorescent tag, which is co-expressed with the capsid gene to identify infected cells. (C) The rep and cap genes required for the AAV production are provided in an additional plasmid. The cap gene used for library search includes 3 silencing sites to prevent expression of AAV9 VP1, VP2 and VP3 capsid proteins.

FIG. 2: Infection of CNS tissue with PHP.eB capsid and GFP transgene (GFP) demonstrates no infection of microglia (Iba-1) as determined by the lack of double positive (yellow) cells.

FIG. 3: Infection of CNS tissue with Random Library. (A) GFP transgene (GFP) demonstrates infection of microglia (Iba-1) as determined by the presence of double positive (yellow) cells. (B) FACS plot of Microglia infection (PE/561) with GFP transgene (GFP/488). Double positive cells (PE⁺/GFP⁺), highlights microglia/brain macrophage cells that had been successfully infected by the virus.

FIG. 4: Characterisation of four novel binding arms for AAV entry into microglia. (A) Representative FACS plot showing the percentage infection rate of microglia using one of the novel viruses containing the random library insertion HGTAASH. (B) Representative FACS plot showing the percentage infection rate of microglia using one of the novel viruses containing the random library insertion ALAVPFR. (C) Representative FACS plot showing the percentage infection rate of microglia using one of the novel viruses containing the random library insertion ALAVPFK. (D) Representative FACS plot showing the percentage infection rate of microglia using one of the novel viruses containing the random library insertion YAFGGEG.

FIG. 5: Microglia isolated and cultured from in vivo infected brains, scale 50 µM.

FIG. 6: Comparison of brain penetration of novel capsid (HGTAASH) against AAV9 and PHP.eB. C57/BL6 mice were injected with an AAV9 encoding a GFP reporter (addgene), a PHP.eB with GFP reporter (addgene) and a novel virus with the binding arm HGTAASH encoding an mCherry reporter (HGTAASH). After injection of 5×10¹¹ virus particles, mice were kept for 4 weeks to allow for protein expression. (a) Injection of AAV-9 demonstrated no uptake throughout the brain parenchyma. (b) Injection of PHP.eB demonstrated uptake in 42% of non-microglia brain cells but no microglia. (c) The HGTAASH capsid. penetrated 17% of non-microglia brain cells and 75% of microglia. (d-f) Immunohistochemistry staining of brain sections of (d) AAV 9 with GFP reporter, (e) PHP.eB with GFP reporter and (f) HGTAASH vector with mCherry reporter (M3 Microglia vector). Iba-1 microglia (white) and associated reporter gene; either GFP (green) or mCherry (red). Scale bar 10uM.

FIG. 7: Gain of function transgene expression in microglia. C57/BL6 mice were injected with a diphtheria toxin (DTA) transgene under control of a human CD11b promoter. (a) Flow cytometry demonstrated a significant reduction of microglia in samples after DTA transgene expression compared to a control mouse injected with a mCherry reporter. (b) Immunohistochemistry confirms reduced numbers of microglia in tissue sections (c) High magnification images of microglia in samples injected with an mCherry reporter identify resting state microglia compared to microglia undergoing apoptosis in samples injected with a virus containing DTA. Scale bars (b) 50uM (c) 10uM.

FIG. 8: Induced down regulation of function in microglia. B6J.129(Cg)-Gt(ROSA)26Sortm1.1(CAG-cas9*,-EGFP) mice were injected with a virus encoding mCherry under the control of the human Cd11b promoter and a control shRNA or an shRNA designed against GFP, both under the control of an U6 promoter. (a) Flow cytometry identified a microglia infection rate of 75% as previously described. Furthermore (b) in mice injected with GFP targeting shRNA 58% of infected microglia had reduced levels of GFP. (c) Immunohistochemistry confirmed a reduction in GFP expression in mCherry positive microglia. Iba-1 microglia (red) and associated reporter gene; either GFP (green) or mCherry (yellow). Scale bar 10uM.

FIG. 9: CRISPR gene editing of microglia. B6J.129(Cg)-Gt(ROSA)26Sortm1.1(CAG-cas9*,-EGFP) mice were injected with a virus encoding a guide RNA against GFP driven by a U6 promoter. (a) Flow cytometry showed a microglia infection rate of 75% as previously reported. Furthermore (b) in mice injected with GFP targeting guide RNA 17% of infected microglia had reduced levels of GFP. (c) Immunohistochemistry confirmed a reduction in GFP expression in mCherry positive microglia. Iba-1 microglia (red) and associated reporter gene; either GFP (green) or mCherry (yellow). Scale bar 10uM.

FIG. 10: In vitro infection of human microglia. Microglia were isolated from human embryos between 8-12 weeks of age. Microglia specific capsid viruses (HGTAASH) encoding an mCherry reporter under the control of human Cd11b promoter were added to the media at 1×10⁹ and infection reates were compared to that of PHP.eB Immunocytochemistry demonstrates mCherry expression in human microglia in culture compared to infection with PHP.eB Scale bar 10uM.

FIG. 11: Random capsid sequences can be inserted into AAV 2 & AAV 6. The novel insertion sequence HGTAASH was inserted into capsid sequences of other AAV serotypes to test possible infectivity of microglia by these engineered capsids. Characterised binding arms capable of infecting microglia were cloned into AAV2 and AAV6. Microglia from C57/BL6 mice were isolated as previously described. AAVs using modified capsid sequences were added to the media at 1×10⁹. Immunocytochemistry demonstrates a modified AAV2 with mCherry transgene expression in murine microglia in culture and modified AAV6 with mCherry transgene expression in murine microglia in culture compared to PHP.eB which is known to readily infect the CNS but not microglia. Scale bar 10uM.

### BRIEF DESCRIPTION OF THE TABLES

Table 1: Table of PCR primers for DNA amplification.

Table 2: Table of designed sequences for incorporation into plasmids.

Table 3: Table showing five additional novel binding arms. Sequences that after insertion into the capsid protein allowed for infection of brain resident microglia were identified by Sanger sequencing and nucleotide sequences were translated in silico. * Denotes the peptide sequence that occurs at high frequency.

Table 4: Table of additional novel binding arm sequences capable of infecting microglia identified using Illumina sequencing.

### BRIEF DESCRIPTION OF THE SEQUENCES

SEQ ID NO: 1 shows the amino acid sequence of insertion sequence 1.

SEQ ID NO: 2 shows the amino acid sequence of insertion sequence 2.

SEQ ID NO: 3 shows the amino acid sequence of insertion sequence 3.

SEQ ID NO: 4 shows the amino acid sequence of insertion sequence 4.

SEQ ID NO: 5 shows the amino acid sequence of insertion sequence 5.

SEQ ID NO: 6 shows the amino acid sequence of insertion sequence 6.

SEQ ID NO: 7 shows the amino acid sequence of insertion sequence 7.

SEQ ID NO: 8 shows the amino acid sequence of insertion sequence 8.

SEQ ID NO: 9 shows the amino acid sequence of insertion sequence 9.

SEQ ID NO: 10 shows the wildtype sequence of PHP.eB.

SEQ ID NO: 11 shows the amino acid sequence of novel capsid protein 1 comprising insertion sequence 1.

SEQ ID NO: 12 shows the amino acid sequence of novel capsid protein 2 comprising insertion sequence 2.

SEQ ID NO: 13 shows the amino acid sequence of novel capsid protein 3 comprising insertion sequence 3.

SEQ ID NO: 14 shows the amino acid sequence of novel capsid protein 4 comprising insertion sequence 4.

SEQ ID NO: 15 shows the amino acid sequence of novel capsid protein 5 comprising insertion sequence 5.

SEQ ID NO: 16 shows the amino acid sequence of novel capsid protein 6 comprising insertion sequence 6.

SEQ ID NO: 17 shows the amino acid sequence of novel capsid protein 7 comprising insertion sequence 7.

SEQ ID NO: 18 shows the amino acid sequence of novel capsid protein 8 comprising insertion sequence 8.

SEQ ID NO: 19 shows the amino acid sequence of novel capsid protein 9 comprising insertion sequence 9.

SEQ ID NO: 20 shows the DNA sequence of novel capsid protein 1 comprising insertion sequence 1.

SEQ ID NO: 21 shows the DNA sequence of novel capsid protein 2 comprising insertion sequence 2.

SEQ ID NO: 22 shows the DNA sequence of novel capsid protein 3 comprising insertion sequence 3.

SEQ ID NO: 23 shows the DNA sequence of novel capsid protein 4 comprising insertion sequence 4.

SEQ ID NO: 24 shows the DNA sequence of novel capsid protein 5 comprising insertion sequence 5.

SEQ ID NO: 25 shows the DNA sequence of novel capsid protein 6 comprising insertion sequence 6.

SEQ ID NO: 26 shows the DNA sequence of novel capsid protein 7 comprising insertion sequence 7.

SEQ ID NO: 27shows the DNA sequence of novel capsid protein 8 comprising insertion sequence 8.

SEQ ID NO: 28 shows the DNA sequence of novel capsid protein 9 comprising insertion sequence 9.

SEQ ID NO: 29 shows a DNA sequence of insertion sequence 1.

SEQ ID NO: 30 shows a DNA sequence of insertion sequence 2.

SEQ ID NO: 31 shows a DNA sequence of insertion sequence 3.

SEQ ID NO: 32 shows a DNA sequence of insertion sequence 4.

SEQ ID NO: 33 shows a DNA sequence of insertion sequence 5.

SEQ ID NO: 34 shows a DNA sequence of insertion sequence 6.

SEQ ID NO: 35 shows a DNA sequence of insertion sequence 7.

SEQ ID NO: 36 shows a DNA sequence of insertion sequence 8.

SEQ ID NO: 37 shows a DNA sequence of insertion sequence 9.

SEQ ID NOs: 38 and 39 show primer sequences that hybridise to PHP.eB replication centre.

SEQ ID NOs: 40 and 41 show primer sequences that hybridise to the 3′ capsid protein of PHP.eB.

SEQ ID NOs: 42 and 43 show primer sequences that hybridise to T2A-GFP.

SEQ ID NOs: 44and 45 show primer sequences that hybridise to PHP.eB backbone.

SEQ ID NOs: 46 and 47 show primer sequences that hybridise to PHP.eB binding arm.

SEQ ID NO: 48 shows the DNA sequence of the random library fragment.

SEQ ID NO: 49 shows the DNA sequence of the silenced PHP.eB capsid protein.

SEQ ID NO: 50 shows the DNA sequence of the split capsid protein comprising the DNA sequence of insertion sequence 9.

SEQ ID NO: 51 shows the DNA sequence of the split capsid protein comprising the DNA sequence of insertion sequence 2.

SEQ ID NO: 52 shows the DNA sequence of the split capsid protein comprising the DNA sequence of insertion sequence 3.

SEQ ID NO: 53 shows the DNA sequence of the split capsid protein comprising the DNA sequence of insertion sequence 1.

SEQ ID NOs: 54 to 207 show the amino acid sequences of additional novel insertion sequences.

SEQ ID NOs: 208 to 361 show the DNA sequences of additional novel insertion sequences.

SEQ ID NO: 362 shows the amino acid sequence of AAV6.

SEQ ID NO: 363 shows the amino acid sequence of AAV2.

SEQ ID NO: 364 shows the amino acid sequence of AAV6 comprising HGTAASH.

SEQ ID NO: 365 shows the amino acid sequence of AAV2 comprising HGTAASH.

### DETAILED DESCRIPTION OF THE INVENTION

As used in this specification and the appended claims, the singular forms “a”, “an”, and “the” include plural references unless the content clearly dictates otherwise. Thus, for example, reference to “a capsid protein” includes “capsid proteins”, reference to “a polynucleotide” includes “polynucleotides”, reference to “a nucleic acid” includes “nucleic acids”, reference to “a promoter” includes “promoters”, reference to “a viral particle” includes two or more such viral particles, and the like.

All publications, patents and patent applications cited herein, whether supra or infra, are hereby incorporated by reference in their entirety.

The present invention concerns AAV capsid proteins that have been modified to improve cell transduction. AAVs comprising the capsid proteins of the invention can be used to deliver targeted gene expression throughout the CNS, as well as to silence the expression and/or activity of genes involved in neurodegeneration.

### AAV Capsid Proteins

The AAV capsid proteins of the present invention comprise a modification compared to a wild-type AAV capsid protein. Such AAV capsid proteins are functional capsid proteins that have the ability to form an AAV viral particle which can infect and/or transduce cells. A functional capsid protein is one that can encapsidate genetic material, enter a cell and transduce the cell with genetic material. In particular, AAV viral particles comprising an AAV capsid protein of the invention are capable of infecting and/or transducing cells throughout the CNS, such as microglia. The skilled person can readily determine if a modified AAV capsid protein is a functional capsid protein using known methods in the art. Exemplary methods for examining the functionality of an AAV capsid protein are described herein.

The AAV capsid proteins of the present invention can be derived from any adeno-associated virus (AAV) or a derivative thereof. As is known to the skilled person, AAV viruses occurring in nature may be classified according to various biological systems.

The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof, including a gene encoding modified capsid protein of invention. These proteins make up the capsid of an AAV viral particle. The AAV capsid proteins of the invention can have modifications in any of VP1, VP2 and/or VP3. In a preferred embodiment, the AAV capsid proteins of the invention have a modification in VP1.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, recently identified from primate brain. The AAV capsid proteins of the invention may be derived from any AAV serotype. The AAV capsid proteins of the invention may be a chimeric capsid protein. For example, the AAV capsid proteins of the invention may comprise amino acid sequences derived from at least one, at least two, or at least three different AAV serotypes. The numbering of the amino acid residues may differ between AAV serotypes.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes, such as cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

In one embodiment of the invention, the AAV capsid proteins of the present invention are derived from any CNS targeting AAV. In a related embodiment of the invention, the AAV capsid proteins of the present invention are derived from AAV serotype 1 (AAV1), AAV serotype 2 (AAV2), AAV serotype 5 (AAV5), AAV serotype 7 (AAV7), AAV serotype 8 (AAV8), AAV serotype 9 (AAV9), AAV serotype rh10 (AAVrh10), retrograde AAV (AAV retrograde) or PHP.eB. It is most preferred that the AAV capsid proteins of the invention are derived from PHP.eB, a derivative of AAV9.

In one embodiment of the invention, the modified AAV capsid protein having an insertion sequence in the binding arm is a wild-type PHP.eB capsid protein having the sequence of SEQ ID NO: 10. The modified AAV capsid protein of the present invention also encompasses variants of SEQ ID NO: 10 that differ in sequence from SEQ ID NO: 10, but retain the ability to form viral particles that can infect and/or transduce microglia or brain macrophages. Such sequences have at least 75%, at least 80%, at least 85%, at least 90%, at least 95%, at least 98% or at least 99% sequence identity to SEQ ID NO: 10.

Percentage sequence identity of variants is preferably measured over the full length of the corresponding portion of SEQ ID NO: 10, or over at least a 400, 500, 600 or 700 contiguous amino acid section of SEQ ID NO: 10 aligned with the variant sequence.

In a preferred embodiment of the present invention, the AAV capsid protein has been modified to insert the amino acid sequence of SEQ ID NO: 1, 2, 3, 4, 5, 6, 7, 8 or 9. The insertion sequences of the present invention also encompass variants of SEQ ID NO: 1, 2, 3, 4, 5, 6, 7, 8 or 9 that differ in sequence from SEQ ID NO: 1, 2, 3, 4, 5, 6, 7, 8 or 9, but retain the ability to form functional AAV capsid proteins when inserted into the AAV capsid, and which form viral particles which infect and/or transduce microglia or brain macrophages. Such insertion sequences may have at least one amino acid substitution, at least two amino acid substitutions, or at least three amino acid substitutions. Exemplary variant sequences include SEQ ID NOs: 147-150, 162-164 and 176-181. Such insertion sequences may further comprise additional amino acids that extend beyond the sequences provided herein. Thus, insertion sequences of the present invention may be at least 7 amino acids in length, at least 8 amino acids in length, at least 9 amino acids in length, or at least 10 amino acids in length.

As explained in the Examples, the inventors have identified additional insertion sequences which retain the ability to form functional AAV capsid proteins when inserted into the AAV capsid, and which form viral particles which infect and/or transduce microglia or brain macrophages. These additional sequences are set out in Table 4 below. Thus, in another aspect of the invention, any of the sequences set out in Table 4 may be used to infect and/or transduce microglia or brain macrophages. For example, an AAV capsid protein of the invention may be modified to insert the amino acid sequence of any of the insertion sequences set out in Table 4 (SEQ ID NOs: 54 to 207).

### Insertion Sites

The AAV capsid proteins of the invention have been modified to insert the amino acid sequence of SEQ ID NO: 1, 2, 3, 4, 5, 6, 7, 8 or 9, or a variant thereof as described above. The amino acid sequence of SEQ ID NO: 1, 2, 3, 4, 5, 6, 7, 8 or 9 or a variant thereof as described above can be inserted into any desired section of an AAV capsid protein. In one exemplary embodiment, the variant has the amino acid sequence of any one of SEQ ID NOs: 147-150, 162-164 and 176-181and is inserted into any desired section of an AAV capsid protein. In the context of infecting and/or transducing microglia or brain macrophages, the amino acid sequence of any of the insertion sequences set out in Table 4 (SEQ ID NOs: 54 to 207) can be inserted into any desired section of an AAV capsid protein.

In a preferred embodiment, the amino acid sequence of SEQ ID NO: 1, 2, 3, 4, 5, 6, 7, 8 or 9, or a variant thereof as described above is inserted between amino acids 588 and 589 of SEQ ID NO: 10. For example, the amino acid sequence of SEQ ID NO: 1, 2, 3, 4, 5, 6, 7, 8 or 9 or any one of SEQ ID NOs: 147-150, 162-164 and 176-181 is inserted between amino acids 588 and 589 of SEQ ID NO: 10. In the context of infecting and/or transducing microglia or brain macrophages, the amino acid sequence of any of the insertion sequences set out in Table 4 (SEQ ID NOs: 54 to 207) can be inserted between amino acids 588 and 589 of SEQ ID NO: 10.

For the avoidance of doubt, the indication that an insertion site is at amino acid X means that the binding peptide is inserted between amino acids X and X+1 (i.e. the binding peptide is inserted after the indicated amino acid).

In another embodiment, the AAV capsid proteins of the invention have been modified to insert the amino acid sequence of SEQ ID NO: 1, 2, 3, 4, 5, 6, 7, 8 or 9, or a variant thereof as described above at an equivalent position in a sequence having at least 75%, at least 80%, at least 85%, at least 90%, at least 95%, at least 98% or at least 99% sequence identity to SEQ ID NO: 10. For example, the amino acid sequence of SEQ ID NO: 1, 2, 3, 4, 5, 6, 7, 8 or 9 or any one of SEQ ID NOs: 147-150, 162-164 and 176-181 is inserted at an equivalent position in a sequence having at least 75%, at least 80%, at least 85%, at least 90%, at least 95%, at least 98% or at least 99% sequence identity to SEQ ID NO: 10. In the context of infecting and/or transducing microglia or brain macrophages, the amino acid sequence of any of the insertion sequences set out in Table 4 (SEQ ID NOs: 54 to 207) can be inserted at an equivalent position in a sequence having at least 75%, at least 80%, at least 85%, at least 90%, at least 95%, at least 98% or at least 99% sequence identity to SEQ ID NO: 10.

The numbering of the amino acid positions described herein correspond to the amino acid positions of the parent AAV sequence. Described herein are the amino acid positions of the parent PHP.eB sequence, which is derived from AAV9. An equivalent position is a position that is equivalent to position 588 and 589 of SEQ ID NO: 10 (i.e. equivalent to position 588 and 589 of unmodified wild-type PHP.eB). The skilled person can readily identify an equivalent position using known methods in the art. In particular, an equivalent position can be identified by sequence alignment methods. For example, an equivalent position can be identified in a sequence by aligning said sequence with the sequence of SEQ ID NO: 10, to identify the equivalent position to positions 588 and 589 of SEQ ID NO: 10. In one exemplary embodiment, an equivalent position is position 487 to 488 of AAV6 (SEQ ID NO: 362). In one exemplary embodiment, an equivalent position is position 586 to 587 of AAV2 (SEQ ID NO: 363). Thus, by way of example, a modified AAV6 and AAV2 capsid protein containing the novel insertion sequence of HGTAASH is shown in SEQ ID NOs: 364 and 365, respectively.

In one exemplary embodiment, the AAV capsid proteins of the invention have the amino acid sequence of SEQ ID NO: 11, 12, 13, 14, 15, 16, 17, 18 or 19.

### Sequence Identity

Sequence identity may be calculated using any suitable algorithm. For example, the PILEUP and BLAST algorithms can be used to calculate identity or line up sequences such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length (W) in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased.

Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787.

One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

### Nucleic Acid

The invention additionally provides nucleic acids encoding the modified AAV capsid proteins of the invention. In some embodiments, the nucleic acids of the invention comprise a 21 nucleotide insertion sequence that encodes for an amino acid sequence of SEQ ID NO: 1, 2, 3, 4, 5, 6, 7, 8 or 9, or a variant thereof as described above.. In one exemplary embodiment, the nucleic acids of the invention comprise a nucleotide sequence of SEQ ID NO: 29, 30, 31, 32, 33, 34, 35, 36 or 37. In a further exemplary embodiment, the nucleic acids of the invention comprise a 21 nucleotide insertion sequence that encodes for a variant of SEQ ID NO: 1, 2, 3, 4, 5, 6, 7, 8, or 9. Examples of such sequences include SEQ ID NOs: 301-304, 316-318 and 330-335 which retain the ability to encode a modified AAV capsid protein of the invention, and which form viral particles which infect and/or transduce microglia or brain macrophages. In the context of infecting and/or transducing microglia or brain macrophages, the nucleic acids of the invention may comprise the nucleotide sequence of any of the insertion sequences set out in Table 4 (SEQ ID NOs: 208 to 361). In some embodiments, the nucleic acids of the invention comprise at least a 21 nucleotide insertion sequence, at least a 24 nucleotide insertion sequence, at least at 27 nucleotide insertion sequences, or at least a 30 nucleotide insertion sequence that encodes for an amino acid sequence of SEQ ID NO: 1, 2, 3, 4, 5, 6, 7, 8 or 9, or a variant thereof as described above.

In a preferred embodiment of the invention, the nucleic acids have the nucleotide sequence of SEQ ID NO: 20, 21, 22, 23, 24, 25, 26, 27 or 28. The nucleic acids of the present invention also encompass variants of SEQ ID NO: 20, 21, 22, 23, 24, 25, 26, 27 or 28 that differ in sequence from SEQ ID NO: 20, 21, 22, 23, 24, 25, 26, 27 or 28, but retain the ability to encode a modified AAV capsid protein of the invention. Such sequences have at least 75%, at least 80%, at least 85%, at least 90%, at least 95%, at least 98% or at least 99% sequence identity to SEQ ID NO: 20, 21, 22, 23, 24, 25, 26, 27 or 28. For example, the nucleic acids may be modified to replace the nucleotide sequence of SEQ ID NO: 29, 30, 31, 32, 33, 34, 35, 36 or 37 with the nucleotide sequence of any of the insertion sequences set out in Table 4 (SEQ ID NOs: 208 to 361).

Percentage sequence identity of variants is preferably measured over the full length of the corresponding portion of SEQ ID NO: 20, 21, 22, 23, 24, 25, 26, 27 or 28 or over a 500, 1000, 1500 or 2000 contiguous nucleotide section of SEQ ID NO: 20, 21, 22, 23, 24, 25, 26, 27 or 28 aligned with the variant sequence.

As is known to the skilled person, amino acid residues may be encoded similarly by multiple nucleotide sequences. Thus, in one embodiment, the invention encompasses degenerate nucleic acids that differ from SEQ ID NO: 20, 21, 22, 23, 24, 25, 26, 27 or 28 in codon sequence due to the degeneracy of the genetic code.

The invention additionally provides a recombinant DNA comprising a nucleic acid of the invention or a variant thereof as described above. The recombinant DNA may be a recombinant AAV vector comprising a nucleic acid of the invention or a variant thereof as described above. Accordingly, such a recombinant AAV vector encodes for a modified AAV capsid protein of the invention.

Typically, nucleic acids of the invention may be incorporated into an AAV genome, in whole or in part, which thus encodes for a modified capsid protein of the invention. Accordingly, such AAV genomes may comprise a nucleic acid sequence of SEQ ID NO: 20, 21, 22, 23, 24, 25, 26, 27 or 28, or a variant thereof as described above.

The AAV genome of the invention may be derived from an AAV having any serotype. In some embodiments, the AAV genome of the invention is derived from a serotype that differs from the serotype of the capsid protein of the invention. It is preferred that the AAV genome is derived from AAV serotype 2 (AAV2) and the modified capsid protein is derived from AAV9.

Such AAV genomes may additionally encode functions needed for the production of an AAV viral particle in host cells. Thus, an AAV genome of the invention may be used to produce an AAV viral particle that incorporates a modified AAV capsid protein of the invention. Naturally occurring AAV viruses are replication-deficient and rely on the provision of helper functions in trans for completion of a replication and packaging cycle. Accordingly, such AAV viral particles may be replication-deficient.

### Viral Particles

As discussed above, the invention additionally provides AAV viral particles. The AAV particle has a shell composed of many capsid proteins. Typically, an AAV viral particle of the invention incorporates a modified capsid protein of the invention. Accordingly, in some embodiments, an AAV particle of the invention comprises a modified AAV capsid protein of the invention having an amino acid sequence of SEQ ID NO: 1, 2, 3, 4, 5, 6, 7, 8 or 9, or a variant thereof as described above. Examples of such variants include SEQ ID NOs: 147-150, 162-164 and 176-181, which retain the ability to form AAV viral particles which infect and/or transduce microglia or brain macrophages. In the context of infecting and/or transducing microglia or brain macrophages, the AAV viral particles of the invention may comprise a modified AAV capsid protein having an amino acid sequence of any of the insertion sequences set out in Table 4 (SEQ ID NOs: 54 to ₂₀₇).In some embodiments, AAV viral particles of the invention may optionally incorporate cargo, which can be delivered to cells. In a preferred embodiment, the AAV viral particles of the invention comprise a modified capsid protein of the invention and an AAV genome. In one aspect, the AAV particle comprises an AAV genome comprising two ITRs. The AAV viral particles of the invention include transcapsidated forms wherein an AAV genome or derivative having an ITR of one serotype is packaged in an AAV capsid protein of the invention. In one embodiment, the AAV particles of the invention comprising a modified capsid protein of the invention may incorporate an AAV genome that encodes a modified AAV capsid protein of the invention. Such AAV viral particles are capable of delivering the viral genome to cells. In an embodiment of the invention the cell is a nerve cell. In an embodiment of the invention the cell is a glial cell, an oligodendrocyte or an astrocyte. In another embodiment of the invention the cell is a microglia or brain resident macrophage, which includes a CNS-infiltrating macrophage derived from recruited monocytes. In a preferred embodiment of the invention the AAV viral particles of the invention deliver the AAV genome to microglia or brain macrophages.

In one embodiment, AAV particles of the invention comprise a modified AAV capsid protein of the invention and further comprise a recombinant polynucleotide encoding a gene of interest, a gene editing construct, an antibody or antigen-binding fragment, or a gene silencing construct for delivery to a cell. The invention additionally provides a host cell comprising an AAV viral particle of the invention.

The AAV viral particles of the invention may also include chemically modified forms bearing ligands adsorbed to the capsid surface. For example, such ligands may include antibodies for targeting a particular cell surface receptor.

### Transduction Efficiency

Viral particles incorporating the AAV capsid proteins of the invention exhibit increased transduction efficiency in cells compared to viral particles incorporating wild-type AAV capsid proteins or AAV capsid proteins lacking the amino acid sequence insertions of the present invention. In an exemplary embodiment, viral particles having the AAV capsid proteins of the invention exhibit increased transduction efficiency in microglia or brain macrophages compared to viral particles incorporating the wild-type AAV capsid proteins or AAV capsid proteins lacking the amino acid sequence insertions of the present invention. For example, the viral particles having the AAV capsid proteins of the invention exhibit increased transduction efficiency in microglia compared to a viral particle having the unmodified AAV capsid protein having the sequence of SEQ ID NO: 10.

The transduction efficiency can be analysed by any suitable standard technique known to the person skilled in the art, for example, using a fluorescent label. The viral particles having the AAV capsid proteins of the invention may exhibit at least 2-fold, at least 5-fold, at least 10-fold, at least 20-fold or at least 50-fold increased transduction efficiency in cells compared to viral particles having the wild-type AAV capsid proteins or AAV capsid proteins lacking the amino acid sequence insertions of the present invention. The viral particles having the AAV capsid proteins of the invention may exhibit at least 1%, 5%, 10%, 20%, 40%, 80%, 90%, 95% or 99% increased transduction efficiency in cells compared to viral particles having wild-type AAV capsid proteins or AAV capsid proteins lacking the amino acid sequence insertions of the present invention.

### Host Cells

The invention additionally provides a host cell comprising the nucleic acid or recombinant DNA disclosed herein. The nucleic acid or recombinant DNA may be provided as plasmids or other episomal elements in the host cell, or alternatively one or more constructs may be integrated into the genome of the host cell.

The host cells of the invention are capable of producing a viral particle of the invention. In order to provide for assembly of the derivatised genome into an AAV viral particle, additional genetic constructs providing AAV and/or helper virus functions will be provided in a host cell in combination with the derivatised genome.

Any suitable host cell can be used to produce the AAV viral particles of the invention. In general, such cells will be transfected mammalian cells but other cell types, e.g. insect cells, can also be used. In terms of mammalian cell production systems, HEK293 and HEK293T are preferred for AAV vectors. BHK or CHO cells may also be used.

### Use of the Modified AAV V Viral Particles

As described above, AAV viral particles of the invention can be used to deliver cargo to cells. In some embodiments, AAV viral particles of the invention comprising a modified AAV capsid protein of the invention can be used to deliver a nucleic acid and/or an AAV genome to a cell. In an embodiment of the invention the cell is a nerve cell. In an embodiment of the invention the cell is a glial cell, an oligodendrocyte or an astrocyte. In another embodiment of the invention the cell is a microglia or brain resident macrophage, which includes a CNS-infiltrating macrophage derived from recruited monocytes. In a preferred embodiment of the invention the cell is a microglia or brain macrophage. In an exemplary embodiment, an AAV capsid protein of the invention is able to target microglia. Thus, the invention encompasses the use of any one of SEQ ID NOs: 1 to 9 or a variant thereof having at least one amino acid substitution, at least two amino acid substitutions, or at least three amino acid substitutions in targeting and/or infecting microglia. For example, the invention encompasses the use of any one of the insertion sequences set out in Table 4 (SEQ ID NOs: 54 to 207) in targeting and/or infecting microglia or brain macrophages.

In one embodiment, the AAV viral particles of the invention comprising a modified AAV capsid protein of the invention optionally comprise a recombinant polynucleotide encoding for a gene of interest, a gene editing construct, an antibody or antigen-binding fragment, or a gene silencing construct, preferably wherein the recombinant polynucleotide is incorporated into an AAV genome in the particle. In a preferred aspect, the AAV viral particles of the invention comprise an AAV genome that comprises the recombinant polynucleotide encoding for a gene of interest, a gene editing construct, an antibody or antigen-binding fragment, or a gene silencing construct and wherein the AAV genome encodes the modified capsid protein of the invention.

In some embodiments, the AAV viral particles of the invention may be used in a method of gene therapy. For example, a target cell, such as a microglial cell, transduced with an AAV viral particle of the invention comprising a recombinant polynucleotide may express a gene of interest, a gene editing construct target, a binding target for an antibody or antigen-binding fragment or a target for gene silencing.

Any gene that is expressed in brain cells, in particular in microglia, and is involved in a neurological disease can be considered a gene of interest. For example, genes of interest may be involved in; neurodegenerative diseases, such as Alzheimer’s disease; neuroinflammatory diseases such as multiple sclerosis; neurodevelopmental disorders such as autism; neuropsychiatric disorders such as schizophrenia; seizure related disorders such as epilepsy; cerebrovascular related disease such as stroke; brain cancers such as glioblastoma; movement disorders such as Parkinson’s disease; neuroinfections such as encephalitis; pain related disorders such as migraine; or an acute brain injury such as traumatic brain injury. Non-restrictive exemplary target genes of interest include TREM2 in the context of phagocytosis, CCL4/CCL3 in the context of cell migration, CD22 in the context of cellular rejuvenation, CSFIR in the context of cell survival. The most detailed example of relevant genes to microglia function in development and disease are listed (Keren-Shaul et al., 2017; Young et al., 2019; Schirmer et al., 2019; Hammond et al., 2019; Masuda et al., 2019; Giersdottir et al., 2019).

In an embodiment of the invention the AAV viral particles of the invention optionally comprise an AAV genome that incorporates a recombinant polynucleotide encoding a gene of interest. A gene of interest may be associated with a neurodegenerative disease, such as Alzheimer’s disease, or a neurodevelopmental disorder, neuropsychiatric schizophrenia or acute brain injury. Non-restrictive exemplary target genes of interest include TREM2, CCL4/CCL3, CD22 and CSF1R. Accordingly, the AAV viral particles of the invention can be used to drive significantly increased gene expression in brain cells, in particular in microglia. Significantly increased expression can be defined as more than about 10 times, 20 times, 50 times, 100 times, 200 times or 300 times the expression of TREM2, CCL4/CCL3, CD22 and CSF1R in cells when compared with wild-type expression of TREM2, CCL4/CCL3, CD22 and CSF1R. Expression of TREM2, CCL4/CCL3, CD22 and CSF1R can be measured by any suitable standard technique known to the person skilled in the art. For example, RNA expression levels can be measured by quantitative real-time PCR. Protein expression can be measured by western blotting or immunohistochemistry.

In an embodiment of the invention the AAV viral particles of the invention optionally comprise an AAV genome that incorporates a recombinant polynucleotide encoding a gene editing construct that is used to edit a target gene sequence in brain cells, in particular in microglia. These genes may be associated with a neurodegenerative disease, such as Alzheimer’s disease, or a neurodevelopmental disorder, neuropsychiatric schizophrenia or acute brain injury. Non-restrictive exemplary target genes of interest for gene editing include TREM2, CCL4/CCL3, CD22 and CSF1R.

A variety of mechanisms to perform gene editing using the AAV viral particles of the invention are encompassed by the present invention. Such mechanisms include endonuclease-based gene editing methods include systems including but not limited to zinc finger nuclease (ZFN), TAL effector nuclease (TALEN), meganuclease (such as MegaTAL) and CRISPR / Cas9. In some embodiments, the AAV viral particles of the invention optionally comprise an AAV genome that incorporates a recombinant polynucleotide encoding a CRISPR guide RNA and a Cas9 protein or derivative or fragment thereof. In a preferred embodiment, the AAV viral particles of the invention optionally comprise an AAV genome that incorporates a recombinant polynucleotide encoding a CRISPR guide RNA complementary to TREM2, CCL4, CCL3, CD22 or CSF1R, and a Cas9 protein or derivative or fragment thereof.

In an embodiment of the invention the AAV viral particles of the invention optionally comprise an AAV genome that incorporates a recombinant polynucleotide encoding an antibody or antigen-binding fragment that binds to a gene product in brain cells, in particular in microglia. These genes may be associated with a neurodegenerative disease, such as Alzheimer’s disease, or a neurodevelopmental disorder, neuropsychiatric schizophrenia or acute brain injury. Non-restrictive exemplary target genes of interest for antibody or antigen-binding fragment binding include TREM2, CCL4/CCL3, CD22 and CSF1R. Accordingly, AAV viral particles of the invention may be used in gene replacement therapy applications.

The term “antibody,” as used herein, includes immunoglobulin molecules comprising four polypeptide chains, two heavy (H) chains and two light (L) chains interconnected by disulfide bonds, as well as multimers thereof (e.g., IgM). The term “antibody,” as used herein, also includes antigen-binding fragments of full antibody molecules, or any derivative thereof, which may contribute to the formation of a “functional antibody”, exhibiting the desired biological activity. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen.

In an embodiment of the invention the AAV viral particles of the invention optionally comprise an AAV genome that incorporates a recombinant polynucleotide encoding a gene silencing construct that is used to silence target gene expression and/or activity in brain cells, in particular in microglia. These genes may be associated with a neurodegenerative disease, such as Alzheimer’s disease, or a neurodevelopmental disorder, neuropsychiatric schizophrenia or acute brain injury. Non-restrictive exemplary target genes of interest for gene silencing include TREM2, CCL4/CCL3, CD22 and CSF1R.

A variety of mechanisms to silence gene expression or activity using the AAV viral particles of the invention are encompassed by the present invention.

The term silencing used herein encompasses diminishing, inhibition or downregulation of gene expression, diminishing, inhibition or downregulation of transcription, diminishing, inhibition or downregulation of translation, and/or diminishing inhibition or downregulation of protein activity. The diminishing, inhibition or downregulation can be direct, or indirect. Methods of determining the level of diminishing, inhibition or downregulation of gene expression, diminishing, inhibition or downregulation of transcription, diminishing, inhibition or downregulation of translation, and/or diminishing inhibition or downregulation of protein activity are known to the skilled person. Examples include in situ hybridisation to determine gene expression, and immunoblotting to determine protein expression. The diminishing, inhibition or downregulation can be complete or partial. Silencing as described herein can be considered to encompass a 10% diminution, inhibition or downregulation, a 20% diminution, inhibition or downregulation, a 30% diminution, inhibition or downregulation, a 40% diminution, inhibition or downregulation, a 50% diminution, inhibition or downregulation, a 60% diminution, inhibition or downregulation, a 70% diminution, inhibition or downregulation, a 80% diminution, inhibition or downregulation, a 90% diminution, inhibition or downregulation, or a 100% diminution, inhibition or downregulation in gene expression, transcription, translation and/or protein activity. Determining the level of inhibition of gene expression can be carried out by the skilled person using methods known in the art.

In some embodiments of the invention, the recombinant polynucleotide can comprise sequences that act to silence gene expression, transcription, translation and/or protein activity. In such embodiments of the invention, the recombinant polynucleotide can comprise a double-stranded RNA, a ncRNA, a shRNA, siRNA, a miRNA, a CRISPR enzyme sequence such as Cas-9, dCas-9, SaCas-9, dSaCas-9, dSaCas-9-KRAB, a guide RNA, zinc-finger proteins (ZFPs), transcription activator-like effector nucleases (TALENs) and/or DREADDs.

### Double-Stranded RNAs

Using known techniques and based on a knowledge of the sequence of the gene to be silenced, double-stranded RNA (dsRNA) molecules can be designed to silence the gene by sequence homology-based targeting of the gene’s RNA. Such dsRNAs will typically be small interfering RNAs (siRNAs), usually in a stem-loop (“hairpin”) configuration, or micro-RNAs (miRNAs). The sequence of such dsRNAs will comprise a portion that corresponds with that of a portion of the mRNA encoding the gene. This portion will usually be 100% complementary to the target portion within the gene’s mRNA but lower levels of complementarity (e.g. 90% or more or 95% or more) may also be used.

### siRNAs

In one embodiment, the silencing mechanism comprises a small interfering RNA (siRNA). An siRNA acts by activating the RNAi-induced suppression complex. The siRNA molecules can be unmodified or modified and are capable of supressing gene expression. They are typically about 15 to 60 nucleotides in length. In some embodiments, the modified siRNA contains at least one 2′0-Me purine or pyrimidine nucleotide such as a 2′0-Me-guanosine, 2′0-Me-uridine, 2′O-Me-adenosine, and/or 2′O-Me-cytosine nucleotide. The modified nucleotides can be present in one strand (i.e., sense or antisense) or both strands of the siRNA. The siRNA sequences may have overhangs or blunt ends.

The modified siRNA may comprise from about 1% to about 100% (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) modified nucleotides in the double-stranded region of the siRNA duplex. In certain embodiments, one, two, three, four, five, six, seven, eight, nine, ten, or more of the nucleotides in the double-stranded region of the siRNA comprise modified nucleotides.

Suitable siRNA sequences can be identified using any means known in the art. Typically, the methods described in Elbashir et al., Nature, 411:494-498 (2001) and Elbashir et al., EMBO J, 20:6877-6888 (2001) are combined with rational design rules set forth in Reynolds et al., Nature Biotech., 22(3):326-330 (2004).

Preferably, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. Alternatively, siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection. In certain other instances, siRNA molecules can be synthesized as a single continuous oligonucleotide fragment, where the self-complementary sense and antisense regions hybridize to form an siRNA duplex having hairpin secondary structure.

### CRISPR and Guide RNAs

In one embodiment, the silencing mechanism encompasses a mechanism of gene silencing by CRISPR (Clustered Regularly Interspaced Short Palindromic Repeats). In one embodiment, the mechanism of gene silencing by CRISPR involves the use of a guide RNA. The guide RNA may comprise a guide RNA sequence and a tracr RNA. The guide RNA sequence is capable of hybridizing to a target sequence in the DNA to be silenced. The tracr RNA is coupled to the guide RNA sequence. The guide RNA hybridises to the site of the allele and targets a CRISPR-Cas enzyme to said site.

In some embodiments, the guide RNA is between 10-30, or between 15-25, or between 15-20 nucleotides in length. In some embodiments one guide RNA is used. In some embodiments two guide RNAs are used. In some embodiments more than two guide RNAs are used.

Preferably the CRISPR-Cas enzyme is a Type II CRISPR enzyme, for example Cas-9 (CRISPR associated protein 9). In some preferred embodiments, the Cas-9 enzyme is SaCas-9.

The enzyme complexes with the guide RNA. In one embodiment, the complex targeted to the DNA sequence will bind by hybridization. In one embodiment, the enzyme is active and acts as an endonuclease to cleave the DNA either via activation of the nonhomologous end-joining or homologous DNA repair pathway, resulting in a blunt end cut or a nick. In one embodiment, the use of guide RNA or RNAs and the CRISPR enzyme results in the deletion of essential elements of the gene to be silenced, resulting in a nonfunctional gene. In some embodiments, the gene is not transcribed. In some embodiments, the gene is not translated.

In another embodiment, the enzyme is targeted to the DNA of the gene to be silenced but the enzyme comprises one or more mutations that reduce or eliminate its endonuclease activity such that it does not edit the mutant allele but does prevent or reduce its transcription. An example of such an enzyme for use in the invention is dCas-9, which is catalytically dead. In a preferred embodiment, the dCas-9 is dSa-Cas9.

In one embodiment dCas-9 is associated with a transcriptional repressor peptide that can knock down gene expression by interfering with transcription. In a preferred embodiment, the transcriptional repressor protein is Krüppel-associated box (KRAB).

In a related embodiment, the enzyme can be engineered such that it is fused to a transcriptional repressor to reduce or disable its endonuclease function. The enzyme will be able to bind the guide RNA and be targeted to the DNA sequence, but no cleavage of the DNA takes place. The mutant allele may be suppressed, for example, by the shutting down of the promoter or blockage of RNA polymerase.

In another embodiment, the transcription repressor may be bound to the tracr sequence. Functional domains can be attached to the tracr sequence by incorporating protein-binding RNA aptamer sequences, as described in Konermann et al (Genome-scale transcriptional activation by an engineered CRISPR-Cas9 complex, Nature, Vol 000, 2014). The transcription repressor-tracr sequence complex may be used to target other moieties to a precise gene location as desired.

In another embodiment, the CRISPR mechanism of silencing involves CRISPR base editors to knock out genes by changing single nucleotides to create stop codons (CRISPR-STOP method (Kuscu et al. 2017)).

In another embodiment, the CRISPR mechanism of silencing involves CRISPR activation mediated upregulation of a gene, said upregulation resulting in the silencing of a target gene as described herein.

### ZFPs

In another embodiment of the invention, the mechanism of silencing encompasses the use of zinc finger proteins (ZFPs, otherwise known as zinc finger nucleases or ZFNs). A ZFP is a heterodimer in which each subunit contains a zinc finger domain and a FokI endonuclease domain. ZFPs constitute the largest individual family of transcriptional modulators known for higher organisms.

### TALENs

In another embodiment of the invention, the mechanism of silencing encompasses the use of transcription activator-like effector nucleases (TALENs). TALENs comprise a non-specific DNA-cleaving nuclease fused to a DNA-binding domain that can be customised so that TALENs can target a sequence of interest to be silenced (Joung and Sander, 2013).

### DREADDS

In another embodiment of the invention, the mechanism of silencing encompasses the use of designer receptor exclusively activated by designer drugs (DREADDs). DREADDs are families of designer G-protein-coupled receptors (GPCRs) built specifically to allow for precise spatiotemporal control of GPCR signalling in vivo that regulate neuronal excitability. The DREADD system has been used to selectively inhibit or activate neuronal electrical activity (Magnus et al., 2019).

### Promoters and Enhancers for Use in the Delivery of Invention

In one embodiment, the viral genome of the AAV particles presented herein comprise at least one control element which provides for the replication, transcription and translation of a coding sequence encoded therein. In a preferred embodiment of the invention, the at least one control element is a cell-type specific promoter and/or enhancer.

In another related aspect, the viral genome comprised in an AAV viral particle of the invention, which comprises a coding region encoding the gene or interest or the gene silencing construct, further comprises a microglia-specific promoter and/or enhancer or a macrophage-specific promoter and/or enhancer. The gene of interest or the gene silencing construct is typically operably linked to a promoter and/or enhancer. The promoter and/or enhancer may be constitutive but will preferably be a microglia-specific or infiltrating brain macrophage-specific promoter and/or enhancer.

By a microglia-specific promoter and/or enhancer, is meant a promoter and/or enhancer that preferentially drives expression, or only or substantially only drives expression in microglial cells e.g. one that drives expression at least two-fold, at least five-fold, at least ten-fold, at least twenty-fold, or at least fifty-fold more strongly in microglia than in any other cell type.

By an infiltrating brain macrophage-specific promoter and/or enhancer, is meant a promoter and/or enhancer that preferentially drives expression, or only or substantially only drives expression in infiltrating brain macrophages e.g. one that drives expression at least two-fold, at least five-fold, at least ten-fold, at least twenty-fold, or at least fifty-fold more strongly in infiltrating brain macrophages than in any other cell type. In a preferred embodiment, the microglia-specific promoter is a promoter derived from TMEM119, CX3CR1 or P2Y12 (P2RY12). In a preferred embodiment, the macrophage specific promoter is a promoter derived from CD11b, CD68, CSF1R or F4/80. A list of microglia related genes are listed (Keren-Shaul et al., 2017; Young et al., 2019; Schirmer et al., 2019; Hammond et al., 2019; Masuda et al., 2019; Giersdottir et al., 2019). In some embodiments, the promoter drives expression in microglia and infiltrating brain macrophages. In one embodiment, the promoter may be a macrophage-specific promoter, which drives expression in microglia. For example, the viral genome comprised in an AAV viral particle of the invention, which comprises a coding region encoding the gene or interest or the gene silencing construct, further comprises the macrophage-specific promoter CD11b.

In another related aspect, the viral genome comprised in an AAV viral particle of the invention, which comprises a coding region encoding the gene or interest or the gene silencing construct, further comprises a localization sequence to further restrict gene expression to distinct cellular compartments such as the nucleus, cell membrane or cytosol. In an exemplary embodiment, the viral genome comprised in an AAV viral particle of the invention, which comprises a coding region encoding the gene of interest or the gene silencing construct, further comprises a nuclear localization signal (NLS), a nuclear exclusion signal (NES), or a membrane targeting signal.

### Non-Human Transgenic Animal

The invention further provides a transgenic animal comprising cells comprising the capsid protein of the invention or the viral particle of the invention. Preferably the animal is a non-human mammal, especially a primate. Alternatively, the animal may be a rodent, especially a mouse; or may be canine, feline, ovine or porcine.

### Pharmaceutical Compositions and Dosages

The AAV capsid protein, nucleic acid or viral particle of the invention can be formulated into pharmaceutical compositions. These compositions may comprise, in addition to the AAV capsid protein, nucleic acid or viral particle, a pharmaceutically acceptable excipient, carrier, diluent, buffer, adjuvant, stabiliser and/or other materials well known to those skilled in the art. Such materials should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer’s Injection, Lactated Ringer’s Injection, Hartmann’s solution. Preservatives, stabilisers, buffers, antioxidants and/or other additives may be included, as required.

For delayed release, the AAV capsid protein, nucleic acid or viral particle may be included in a pharmaceutical composition which is formulated for slow release, such as in microcapsules formed from biocompatible polymers or in liposomal carrier systems according to methods known in the art.

Dosages and dosage regimes can be determined within the normal skill of the medical practitioner responsible for administration of the composition. The dosage of the active agent(s) may vary, depending on the reason for use, the individual subject, and the mode of administration. The dosage may be adjusted based on the subject’s weight, the age and health of the subject, and tolerance for the compound(s) or composition.

### Methods of Therapy and Medical Uses

The AAV viral particles of the invention may be used in the treatment of a subject. The terms “patient” and “subject” may be used interchangeably. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human. The subject may be male or female.

The subject is preferably identified as being at risk of, or having, a neurological disease. Example categories and examples not limited to include; neurodegenerative disease, such as Alzheimer’s disease; neuroinflammatory diseases such as multiple sclerosis; neurodevelopmental disorders such as autism; neuropsychiatric disorders such as schizophrenia; seizure related disorders such as epilepsy; cerebrovascular related disease such as stroke; brain cancers such as glioblastoma; movement disorders such as Parkinson’s disease; neuroinfections such as encephalitis; pain related disorders such as migraine; or an acute brain injury such as traumatic brain injury.

The terms “treat,” “treated,” “treating,” or “treatment” as used herein refer to both therapeutic treatment and prophylactic or preventative measures, wherein the object is to prevent or slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

The AAV viral particles of the invention may be used in the treatment or prevention of neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury. This provides a means whereby the degenerative process of the disease can be treated, arrested, palliated or prevented.

The invention therefore provides a pharmaceutical composition comprising the AAV viral particles of the invention and a pharmaceutically acceptable carrier.

The invention also provides the AAV viral particles of the invention for use in a method of preventing or treating neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury.

The invention also provides the use of the AAV viral particles of the invention in the manufacture of a medicament for the treatment or prevention of neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury.

The invention also provides a method of treating or preventing neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury, in a patient in need thereof comprising administering a therapeutically effective amount of the AAV viral particles of the invention to the patient.

By using the AAV viral particles of the invention, which comprise a modified AAV capsid protein of the invention, expression of a gene of interest, a gene editing construct, an antibody or antigen-binding fragment, or a gene silencing construct can be achieved throughout cells in the CNS and in particular in microglia and infiltrating brain macrophages. Thus, the AAV viral particles of the present invention can be used to treat or prevent neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury, by expressing a gene of interest, a gene editing construct, an antibody or antigen-binding fragment, or a gene silencing construct throughout cells in the CNS and in particular in microglia and infiltrating brain macrophages, and thus treating the microglia and infiltrating brain macrophages of such patients.

In general, parenteral routes of delivery of the AAVs of the invention, such as intravenous (IV) or intracerebroventricular (ICV) administration, typically by injection or infusion, are preferred.

The invention therefore also provides a method of treating or preventing neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury, in a patient in need thereof, comprising administering a therapeutically effective amount of the AAV viral particles of the invention to the patient by a parenteral route of administration. Accordingly, neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury are thereby treated or prevented in said patient.

In a related aspect, the invention provides for use of the AAV viral particles of the invention in a method of treating or preventing neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury, by administering said AAV viral particles to a patient by a parenteral route of administration. Additionally, the invention provides the use of the AAV viral particles of the invention in the manufacture of a medicament for treating or preventing neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury, by a parenteral route of administration.

In all these embodiments, the AAV viral particles of the invention may be administered in order to prevent the onset of one or more symptoms of neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury. The patient may be asymptomatic. The subject may have a predisposition to the disease. The method or use may comprise a step of identifying whether or not a subject is at risk of developing, or has, a neurodegenerative disease, a neuroinflammatory disease, a neurodevelopmental disorder, a neuropsychiatric disorder, a seizure related disorder, a cerebrovascular related disease, a brain cancer, a movement disorder, a neuroinfection, a pain related disorder, or an acute brain injury. A prophylactically effective amount of the AAV is administered to such a subject. A prophylactically effective amount is an amount which prevents the onset of one or more symptoms of the disease.

Alternatively, the AAV viral particles of the invention may be administered once the symptoms of the disease have appeared in a subject i.e. to cure existing symptoms of the disease. A therapeutically effective amount of the antagonist is administered to such a subject. A therapeutically effective amount is an amount which is effective to ameliorate one or more symptoms of the disease.

The subject may be male or female. The subject is preferably identified as being at risk of, or having, the disease.

The administration of the AAV viral particles of the invention is typically by a parenteral route of administration. Parenteral routes of administration encompass intravenous (IV), intramuscular (IM), subcutaneous (SC), epidural (E), intracerebral (IC), intracerebroventricular (ICV), intranasal (IN) and intradermal (ID) administration.

The dose of the AAV viral particles of the invention may be determined according to various parameters, especially according to the age, weight and condition of the patient to be treated; the route of administration; and the required regimen. Again, a physician will be able to determine the required route of administration and dosage for any particular patient. For example, a suitable dose of an AAV of the present invention may be in the range of about 1 ×10⁶ vg to about 1 ×10¹⁶ vg, where vg = viral genome. In some embodiments, a suitable dose of an AAV of the present invention may be in the range of about 1 ×10⁶ vg, about 1 ×10⁷ vg, about 1 ×10⁸ vg, about 1 ×10⁹ vg, about 1 ×10¹⁰ vg, about 1 ×10¹¹vg, about 1 ×10¹² vg, about 1 ×10¹³ vg, about 1 ×10¹⁴ vg, about 1 ×10¹⁵ vg, or about 1 ×10¹⁶vg.

Any suitable dosing schedule for administering the AAV of the invention may be used, including single dosing or multiple dosing regimens, such as split dosing regimens.

### Combination Therapies

The capsid proteins, nucleic acids, viral particles and/or pharmaceutical compositions can be used in combination with any other therapy for the treatment or prevention of neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury.

In one exemplary embodiment, the capsid proteins, nucleic acids, viral particles and/or pharmaceutical compositions can be used in combination with an immunosuppressant to mediate the immune response against AAV.

Compositions can be administered concurrently with, prior to, or subsequent to, one or more other desired therapies for the treatment or prevention of neurodegenerative diseases, neuroinflammatory diseases, neurodevelopmental disorders, neuropsychiatric disorders, seizure related disorders, cerebrovascular related diseases, brain cancers, movement disorders, neuroinfections, pain related disorders, or an acute brain injury.

### Diagnostic Methods

The capsid proteins, nucleic acids or viral particles of the invention can be used in a diagnostic method. In one exemplary embodiment, the capsid proteins, nucleic acids or viral particles of the invention may be used in diagnostic methods using positron emission tomography (PET) imaging. For example, the capsid proteins, nucleic acids or viral particles of the invention may be used in a PET study of systemic AAV capsid accumulation and clearance in the brain following parenteral administration.

### Methods of Targeting Microglia

The invention also relates to a method of targeting cells using an AAV capsid protein of the invention. In one embodiment, the invention provides a method of targeting microglia using an AAV capsid protein of the invention, the method comprising introducing a recombinant AAV vector into a mammal, the recombinant AAV vector encoding for a gene of interest or a silencing construct as described herein, which is encapsidated into a capsid comprising a capsid protein of the invention.

### Kits

The capsid proteins, nucleic acids, recombinant DNA, viral particles and/or pharmaceutical compositions of the invention can be packaged into a kit.

The following Examples illustrate the invention.

### EXAMPLES

### Materials and Methods

### Generation of In Vivo Plasmid Libraries

AAV-CMVc-Cas9 was a gift from Juan Belmonte (Addgene plasmid # 106431). pCAG-Cre-IRES2-GFP was a gift from Anjen Chenn (Addgene plasmid # 26646). pUCmini-iCAP-PHP.eB was a gift from Viviana Gradinaru (Addgene plasmid # 103005). pHelper plasmid was a gift from the Cancer Research United Kingdom viral core facility. The inventors PCR amplified the appropriate sequences using the primers listed in table 1. Using the AAV-CMVc-Cas9 plasmid backbone and AAV2 ITR sequences, the cap gene and fragments of the rep genes from iCAP-PHP.eB as well as the GFP sequence from Cre-IRES-GFP, the inventors cloned the novel selection library plasmid with a GFP reporter using NEBBuilder HiFi DNA Assembly. The inventors silenced the translation of VP1, VP2 and VP3 encoded in the PHP.eB capsid plasmid by inserting premature STOP codons into the respective coding sequences. The silenced sequence was created and cloned as previously described (Table 2) using NEBBuilder HiFi DNA Assembly. The silenced sequence was created by Integrated DNA Technologies and cloned as previously described (Table 2). The random library was created by Sigma (Table 2).

Viral genomes were all constructed using homology based cloning methods using the DNA-HIFI Builder kit (NEB). Human Cd11b promoter was cloned from human DNA isolated from buccal cells. DTA was cloned from genomic DNA of a transgenic mouse carrying the transgene. dsDNA for the gRNA and shRNA sequences were created by oligo annealing of ssDNA oligos from Sigma. KASH domain was cloned from MEF cDNA. The gRNA sequence targeting GFP was sub-cloned into a construct with U6 promoter by Golden Gate cloning using BsaI-HFv2 (NEB). The shRNA sequences were subcloned into the shRNA vector by restriction enzyme cloning. The modified capsid sequences of AAV2 and AAV6 were created by PCR. Primers were created flanking exactly the insertion site of the newly identified sequences. The sequences were then added as extensions on the 5′ of each primer in a fashion that there was at least a 20bp overlap between the inserted sequences. PCRs were performed using Q5 polymerase effectively creating a linear dsDNA fragment of the vector of which the 5′ and 3′ shared a 20bp homologous sequence that included the newly added sequences. PCR products were purified by column purification and 10 ng of purified DNA were transformed into NEB DH5-alpha using standard protocols. In all instances positive clones were confirmed by Sanger sequencing.

### Virus Production Protocol and in In Vivo Transfection

AAV production and purification followed the previously described protocol (Challis et al., 2018). In brief, HEK293 cells were grown in 15 mm plastic dishes and triple transfected with the pHelper plasmid, the silenced PHP.eB capsid plasmid, and the transfer plasmid encoding for the capsid library plasmid. Five days later cells were lysed and virus precipitated using PEG. Viral particles were purified using Optiprep density gradient medium (Sigma; D1556) and ultra-centrifugation at 350000 g. Viral layer was isolated and concentrated using Amicon Ultra-15 Centrifugal Filter Units (Sigma; Z648043-24EA). AAV titer was determined using SYBR green qPCR. For in vivo administration of the virus, 8-week old mice were restrained and 5×10¹¹ viral genomes per virus were injected into the tail vein.

### Immunofluorescence for Tissue Sections

Mice received a lethal dose of pento-barbitol and were transcardially perfused with 4% paraformaldehyde (PFA) in PBS. The brains were removed and post-fixed for 2 h at RT with 4% PFA. After a rinse in PBS the tissue was incubated in 20% sucrose solution (in PBS) overnight. The tissue was then imbedded in OCT- medium (TissueTek) and stored at -80° C. 12 µm sections were obtained using a cryostat. Tissue sections were air-dried and stored at -80° C. Cryostat cut sections were dried for 45 min at RT. The slides were washed three times with PBS (5 min, RT) and blocked in 0.3% PBST with 10%NDS for 1 h at RT. Primary antibodies were diluted in 0.1% PBST with 5%NDS and incubated overnight at 4° C. The slides were washed 3 times for 10 min with PBS. Next, secondary antibodies in blocking solution were applied at a concentration of 1:500 for 2 h at RT. Slides were washed 3 times with PBS for 10 min each, whereby the first wash contained Hoechst 33342 nuclear stain (2 µg/ml,). The slides were mounted with coverslips using FluoSave (CalBiochem). Image acquisition was performed using a Leica-SP5 microscope (Leica) and LAS software (Leica) or a Zeiss Observer A1 inverted microscope (Zeiss) and Zeiss Axivision software. Further image processing and analysis was performed using the ImageJ software package.

### Isolation of Single Cell Suspension

Adult male and female mice (8 weeks) were decapitated after lethal injection with phenobarbital. The brains were removed quickly and placed into ice-cold isolation medium. The telencephalon and cerebellum were dissected in isolation medium; meninges, and the olfactory bulb were mechanically removed and the brain tissue was mechanically minced into 1 mm³ pieces. The tissue pieces were spun down at 100 g for 1 min at RT and the tissue was washed in HBSS- (no Mg2+ and Ca2+, GIBCO). Each brain was mixed with 5ml of dissociation solution (34U/ml papain (Worthington), 20 µg/ml DNase Type IV (GIBCO) in isolation medium (in house made Hibernate-A for low fluorescence (Brainbits). The brain tissue was dissociated on a shaker (50 rpm) for 30 min at 35° C. The digestion was stopped by addition of ice cold HBSS-. The tissue was centrifuged (200 g, 3 min, RT), the supernatant completely aspirated and the tissue resuspended in isolation medium supplemented with 2% B27 and 2 mM sodium-pyruvate (trituration solution). The tissue was allowed to sit in this solution for 5 min. To obtain a single cell suspension the tissue suspension was triturated 10 times using first a 5 ml serological pipette and subsequently three fire polished glass pipettes (opening diameter > 0.5 mm). After each trituration step the tissue suspension was allowed to sediment (approximately 1-2 min) and the supernatant (approximately 2 ml), containing the cells, was transferred into a fresh tube. After each round of trituration 2 ml of fresh trituration solution were added. To remove accidentally transferred undigested tissue bits, the collected supernatant was filtered through 70 µm cell strainers into tubes that contained 90% isotonic Percoll (GE Healthcare, 17-0891-01, in 10xPBS pH7.2 (Lifetech). The final volume was topped up with phenol-red free DMEM/F12 with HEPES (GIBCO) and mixed to yield a homogeneous suspension with a final Percoll concentration of 22.5%. The single cell suspension was separated from remaining debris particles by gradient density centrifugation (800 g, 20 min, RT, without break). The myelin debris and all layers without cells were discarded and the brain cell containing phase (last 2 ml) and cell pellet were resuspended in HBSS+ and combined in a fresh 15 ml tubes and centrifuged (300 g, 5 min, RT). The cell pellet was resuspended in red blood cell lysis buffer (Sigma, R7757) and incubated for 1 min at RT to remove red blood cells. 10ml of HBSS+ were added to this cell suspension and spun down (300 g, 5 min, RT). The cell pellets were resuspended in 0.5ml modified Milteny washing buffer (MWB, 2 mM EDTA, 2 mM Na-Pyruvate, 0.5% BSA in PBS, pH 7.3) supplemented with 10 ng/ml human recombinant insulin (GIBCO).

### Fluorescence Activated Cell Sorting

Freshly isolated cells from the brain were stained with primary antibodies (Anti-CD11b-PE and appropriate isotype controls) for 15 min at 4° C. Cells were washed and resuspended in FACS buffer. Cells were analyzed using an Attune-NXT (Thermo Scientific) equipped with 405, 488 and 561 lasers. For compensation, beads (OneComp) were used for single stains for the fluorophore. The compensation matrix was automatically calculated and applied by the Attune software. Gates for the quantification of Cd11b and GFP positive cells were set according to appropriate FMOs. A minimum of 50,000 cell singlets were recorded and used for quantification with FlowJo software (v10).

### Culturing of Human and Mouse Microglia

Isolated microglia are seeded into 96 well plate at a density of 1 cell per well. DMEM F12 with 60 µg/ml N-Acetyl cysteine (Sigma), 10 µg/ml human recombinant insulin (GIBCO), 1 mM sodium pyruvate (GIBCO), 50 µg/ml apo-transferrin (Sigma), 16.1 µg/ml putrescine (Sigma), 40 ng/ml sodium selenite (Sigma), 20 ng/ml M-CSF. 330ug/ml Bovine serum albumin (Sigma) can be added if cells are struggling to survive however, this will reduce their proliferative potential. Microglia are incubated at 37° C., 5% CO2 and 5% O2. After 10 days 50% of the medium is then exchanged for media with 10 ng/ml M-CSF. After which media should be changed every 3 days.

### Next Generation Sequencing for Identification of Capsid Sequences

Cells were collected by FACS and pelleted by centrifugation. The supernatant was removed to a volume of approximately 20µl and 50µl of lucigen quick DNA extract solution was added to all samples. The samples were then processed according to the manufacturer’s instructions. Sequencing libraries were then created by two rounds of PCR adding the unique barcodes, flow-cell annealing barcodes and illumina indexe sequences. In the first round of PCR we used 5µl of the DNA containing quick extract solution in a 50µl PCR reaction. The PCR product was cleaned up using 1.8X AmpureXP beads and eluted in 40µl. 5% of this eluate was used as a template in the second round of PCR. For all PCRs Q5 polymerase (NEB) was used. The product was cleaned up using AmpureXP beads (0.5X). Libaries were then quality assessed by qPCR and using a bioanalyser. Pooled libraries were spiked with 10% PhiX and sequenced using a Illumina Nano (2×250bp) kit on a Illumina MiSeq.

### Example 1: PHP.eB Cannot Infect Microglia in an In Vivo Setting

To identify whether PHP.eB was capable of infecting microglia cells, the inventors generated an AAV encoding for spCas9 using the PHP.eB capsid and injected C57/B6 mice. Mice were perfusion fixed after 21 days and the brain tissue was stained with an anti HA-tag antibody capable of detecting the HA-tagged Cas-9 enzyme (Segel et al., 2019). In order to confirm that this was not associated with the ability of microglia to reject Cas9 entry into the cell, the inventors repeated the experiment with the injection of an AAV expressing green fluorescent protein (GFP) under control of a CMV promoter using the PHP.eB capsid. Tissues were stained with Iba-1 antibody to detect microglia within the tissue. Full brain sections were examined for the presence of double labelled GFP expressing Iba-1 labelled cells. No double labelled cells were identified across the tissue (n=3) (FIG. 2). These results were in keeping with the original article from Deverman et al., (2016), where data was presented on the majority of cells groups within CNS with the exclusion of microglia.

### Example 2: Generation of a Random Library to Identify Candidates for Microglia Infection

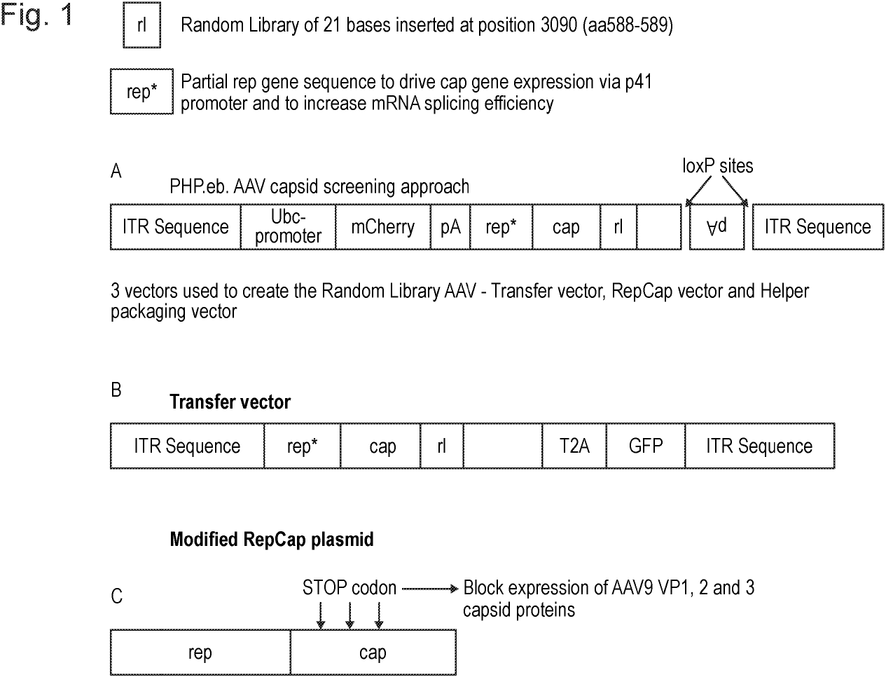

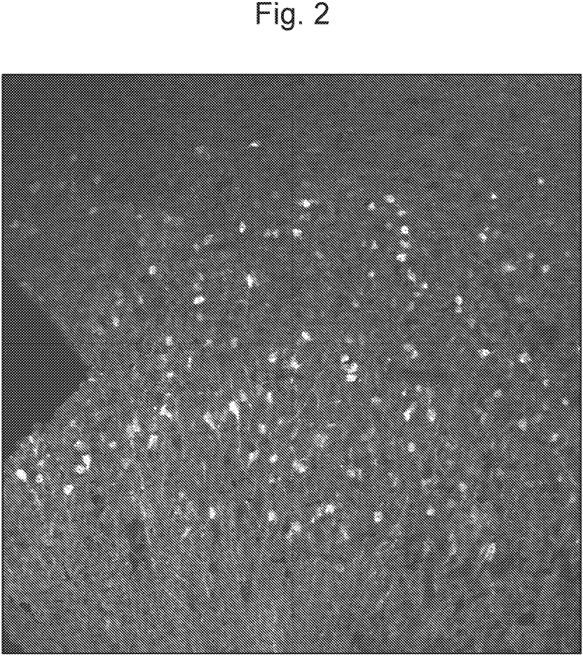

In order to examine whether an AAV was capable of infecting microglia via systemic circulation administration, the inventors created a novel random library of viruses to perform a selection screen. To do this, the PHP.eB capsid plasmid (FIG. 1A) was deconstructed using PCR to isolate individual fragments of interest. For the purposes of the random library screen the PHP.eB capsid sequences of interest were required to be inserted in to a novel transgene plasmid with a fluorescent label in order to identify positive entry. The inventors extracted the backbone and ITR sequences from AAV-CMVc-Cas9 using restriction enzyme digestion (Segel et al., 2019). An expression cassette containing a p41 promoter and rep fragments driving the expression of the capsid library t2a GFP followed by a poly-A tail initiation sequence was constructed by PCR and assembled by homology cloning using NEBuilder HIFI into the novel transfer plasmid. In order to integrate the random library sequence into the binding arm of the virus, the inventors amplified the capsid sequence in two sections excluding the PHP.eB binding arm (amino acid sequence: TLAVPFK (starting from amino acid 589)) and replaced the 21 nucleotides encoding for this sequence with a random sequence library (FIG. 1B).

The PHP.eB capsid plasmid encoding for rep and cap sequences was used as the rep-cap plasmid to generate the AAV. However, to eliminate capsid protein expression the inventors inserted in-frame stop codon with the reading frame for each capsid protein VP1-3 (FIG. 1C) as previously described (Deverman et al., 2016). These stop codons do not alter the amino acid sequence of the assembly activating protein (AAP) which is expressed in an alternative reading frame within the Cap gene (Sonntag et al., 2010). These plasmids were used with the pAAV helper plasmid to create the here described novel AAVs.

### Example 3: Microglia Can Be Systemically Infected Using AAV Technology

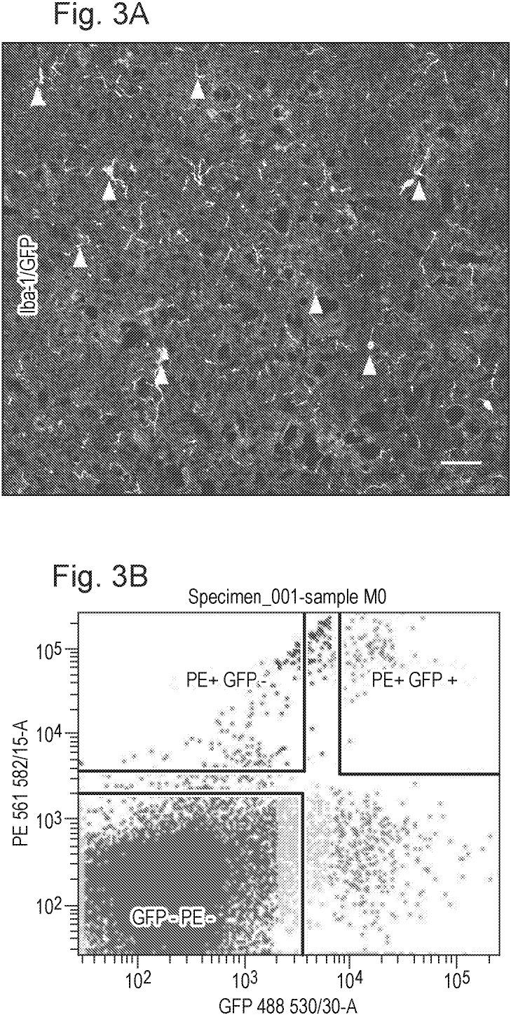

Virus library was injected into the of C57/B6 mice at a concentration of 5×10¹¹ viral genomes in 150µl sterile PBS. 21 days later mice were sacrificed and had the whole brain removed for the purposes of creating a single cell suspension or were perfusion fixed in order to perform immunohistochemical staining. Immunohistochemical staining of Iba-1 demonstrated double positive microglia in indicative of successful infection (FIGS. 3A and B ). For the purposes of sorting positive GFP labelled cells fresh brains were homogenised into a single cell suspension using established techniques [Neumann et al., 2019] and labelled with a CD11b/PE FACS antibody. Double positive cells (CB11b⁺/GFP⁺) were sorted (FIG. 3C) and the DNA extracted.

### Example 4: Novel Binding Arms of AAV9/PHP.eB Allow for Microglia Entry

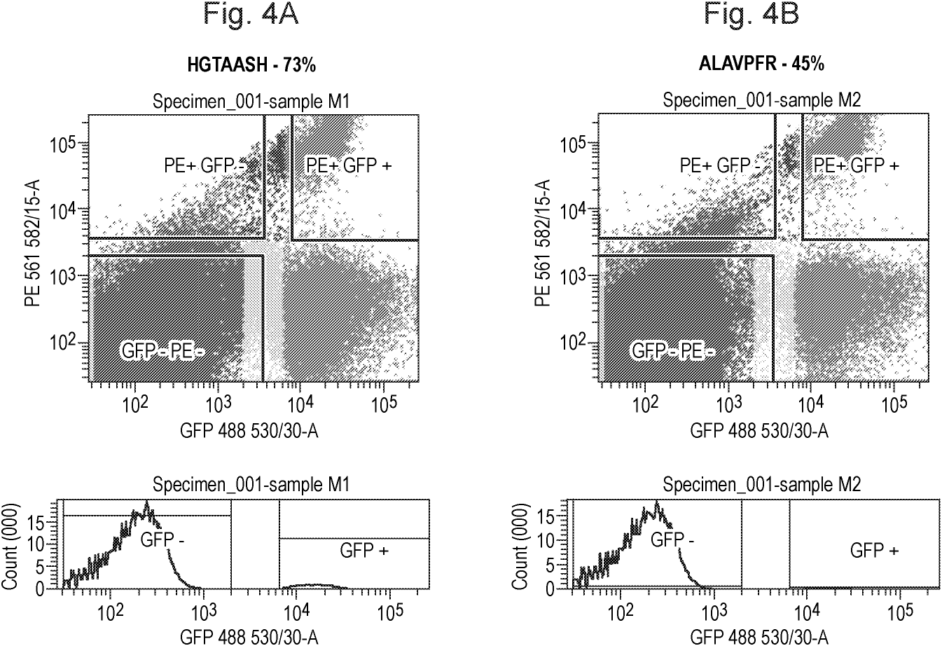

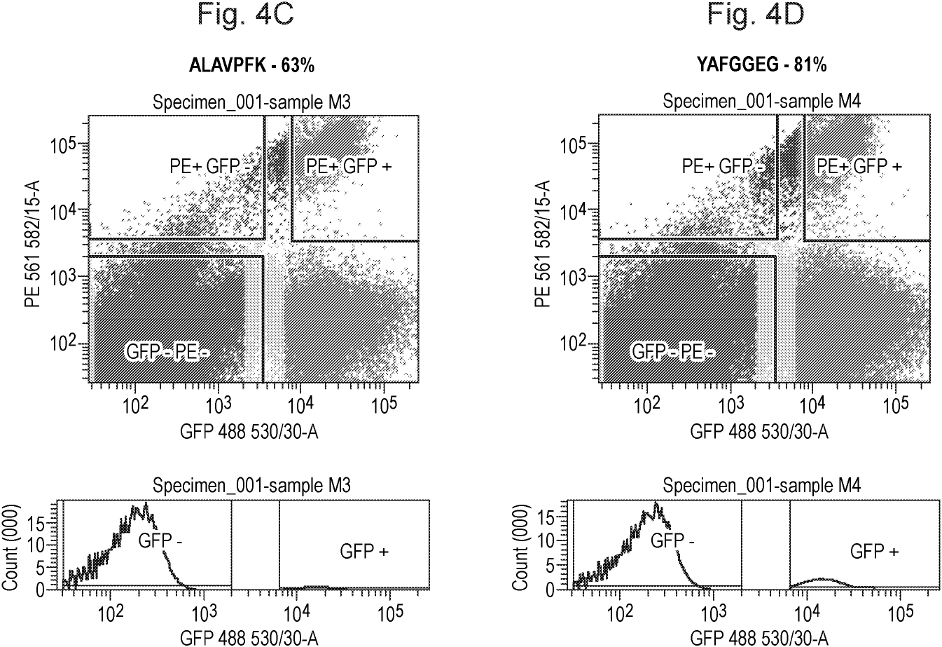

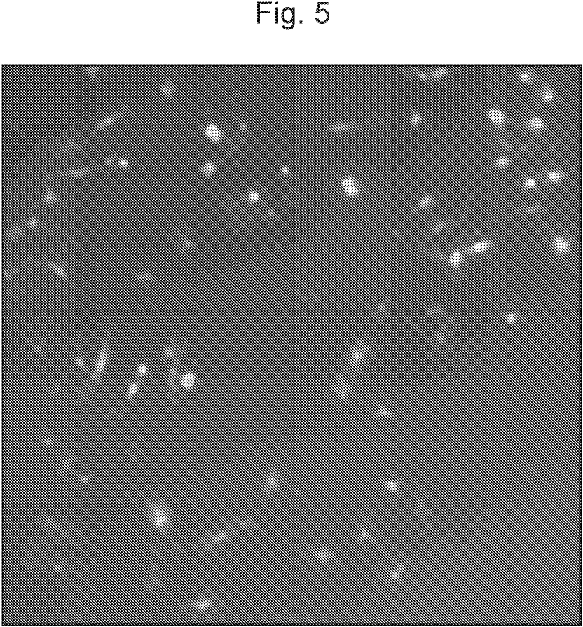

DNA from GFP positive microglia was isolated and a viral genome region including the random library sequence was PCR amplified. PCR products were subcloned using TOPO cloning. DNA from individual bacterial colonies was extracted and a region including the random library sequence was sequenced using Sanger sequencing. Nine sequences were identified by TOPO cloning (FIGS. 4A-D and Table 3). Of these, four sequences were used to create novel capsid plasmids as described and combined with a GFP expressing transfer plasmid for the purpose of characterisation each novel binding arm. Each of the four novel viruses were injected into the tail vein of C57/B6 mice at a concentration of 5×10¹¹ which were culled after 21 days. Immunohistochemical analysis of the tissue demonstrated double positive labelling of Iba-1 positive microglia with GFP. To further confirm this finding, the inventors isolated microglia using CD11b beads via MACS and cultured them to determine viability (FIG. 5). Finally, FAC sorting of CD11b⁺ microglia identified the percentage of GFP positive microglia, which varied between 45-81% using the four novel viruses (FIGS. 4A-D ).

### Example 5: Generation of Viruses

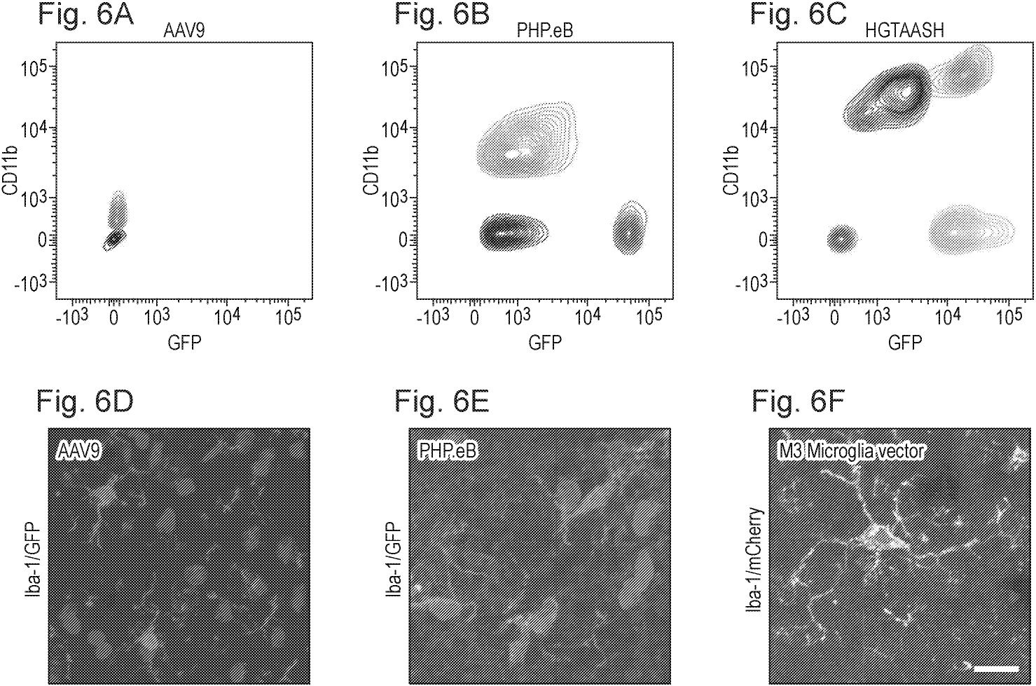

Microglia specific viruses were generated by using the CD11b promoter together with an mCherry fluorescent tag, localized to the perinuclear lamina. Whilst the inventors recognize that CD11b is proposed as a pan macrophage marker, their single cell sequencing data has demonstrated that in the absence of an acute brain injury, CD11b is an excellent marker for isolating microglia from the brain parenchyma (Young et al., 2021). Three specific microglia viruses were created, with appropriate controls and injected into the tail vein of C57/B6 mice at a concentration of 5×10¹¹ and incubated for 4 weeks. Specifically, brain and microglia penetration of AAV9, PHP.eB and one of the novel capsid sequences containing an insertion sequence of the invention (HGTAASH) was compared. Succesful infection was determined by observing the expression of transgenes in cells that were labelled with the microglia marker Iba1 by IHC. Injection of AAV 9 demonstrated no uptake throughout the brain parenchyma at the concentration and imaging settings used (FIG. 6a). Injection of PHP.eB demonstrated uptake in 42% of non-microglia brain cells but no transgene expression was observed in microglia (FIG. 6b). The novel microglia capsid penetrated 17% of non-microglia brain cells and 75% of microglia (FIG. 6c). Immunohistochemical staining of brain sections confirmed that the novel microglia capsid penetrated microglia, which was not observed in AAV9 or PHP.eB control samples (FIGS. 6d-f ).

### Example 6: Upregulation of Transgene in Microglia In Vivo

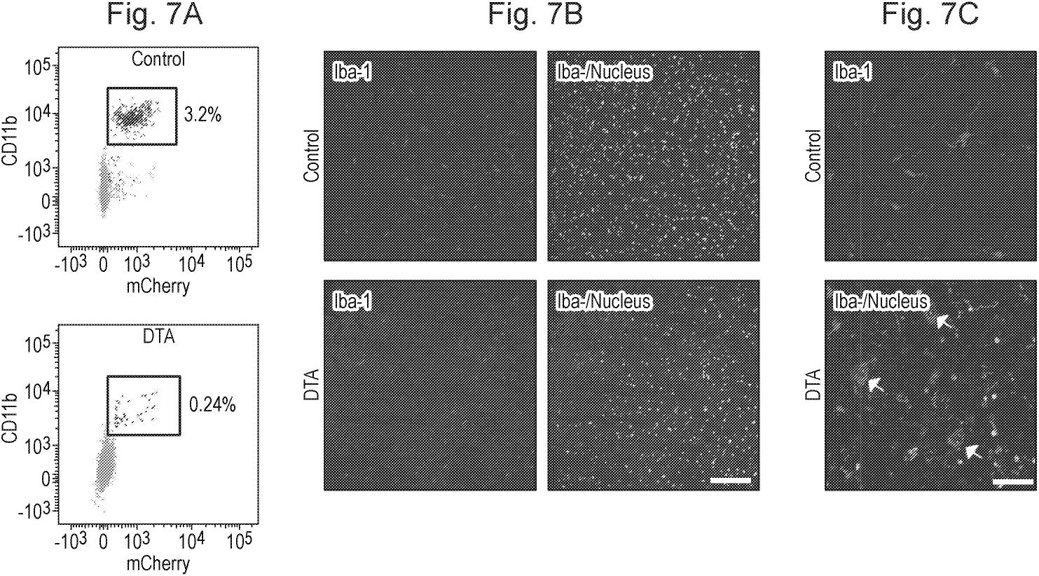

To assess if the overexpression of a transgene could produce a measureable phenotypic effect in microglia, C57/BL6 mice were injected with 5×10¹¹ viral particles encoding for the diphtheria toxin (DTA) transgene under control of a human CD11b promoter. Expression of DTA within cells results in cell death as a result of toxicity. As such, microglia expressing Cd11b, but not other brain cell types, would undergo apoptosis which would result in a depletion of microglia in the brain. A significant reduction of microglia in samples after DTA transgene expression compared to controls was observed using flow cytometry. Specifically, microglia numbers were reduced to 0.24% from 3.2% (FIG. 7a) as measured by flow cytometry. Immunohistochemistry confirmed large areas of tissue with depleted microglia (FIG. 7b) and more interestingly, large numbers of Iba1 positive cells undergoing active apoptosis (FIG. 7c).

### Example 7: Downregulation of Gene Expression in Microglia In Vivo

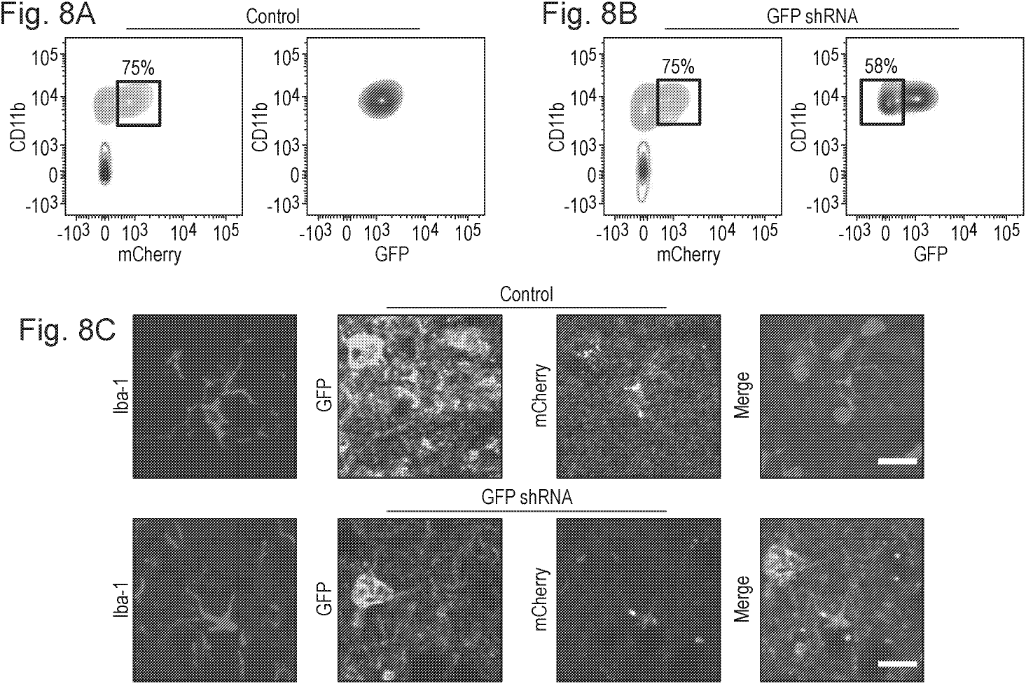

The inventors next addressed if the newly generated virus capsids would allow infection of microglia with constructs that would interfere with gene expression of host microglia. B6J.129(Cg)-Gt(ROSA)26Sortm1.1(CAG-cas9*,-EGFP) mice were injected with 5×10¹¹ viral particles encoding for an shRNA directed against GFP driven by a U6 promoter and a CD11b promoter controlling mCherry-KASH domain fusion protein expression. Positively infected cells express mCherry around the nuclear lamina and those cells successfully expressing a shRNA against GFP, which is expressed by all cells in the CAG-Cas9-EGFP mice, should have reduced or no expression of GFP in microglia compared to microglia from mice that were infected with a control virus in which the shRNA is a scrambled control not targeting any gene (FIG. 8a). Flow cytometry identified microglia infection of 75% as previously described (see FIG. 6; Example 5). Furthermore in mice injected with GFP targeting shRNA, 58% of infected microglia had reduced levels of GFP compared to mice injected with a control shRNA (FIG. 8b). Immunohistochemistry confirmed a reduction in GFP expression in mCherry positive microglia (FIG. 8c).

### Example 8: CRISPR Gene Editing of Microglia

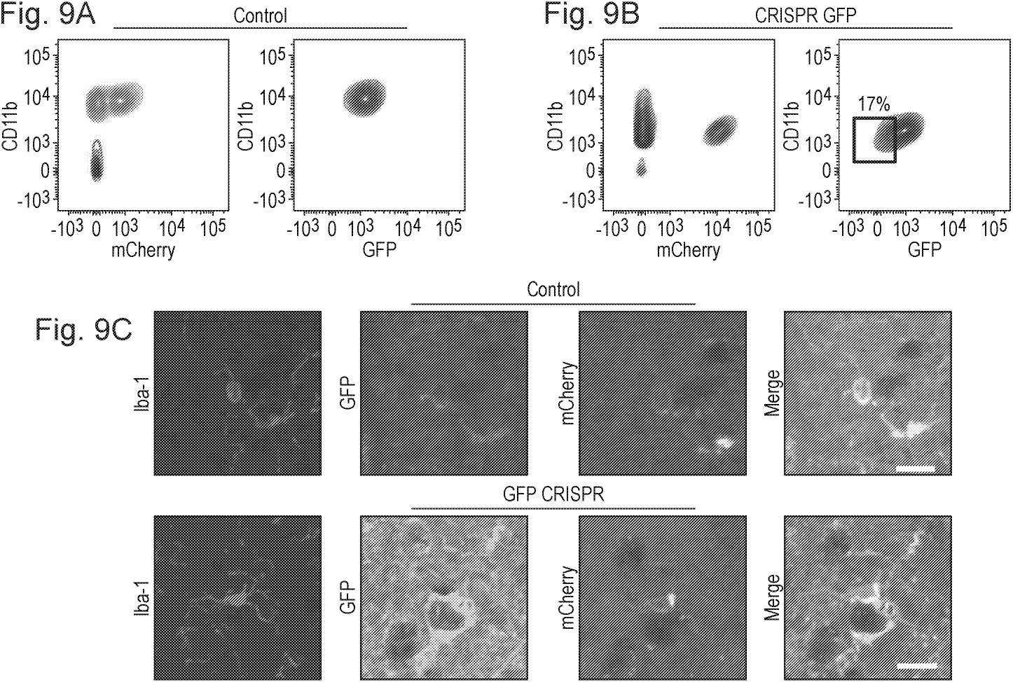

In order to determine whether the genome of microglia could be edited using CRISPR methods, B6J.129(Cg)-Gt(ROSA)26Sortm1.1(CAG-cas9*,-EGFP) mice were injected with 5×10¹¹ viral particles encoding for a gRNA against GFP. To control for infection, the virus contained a second expression cassette encoding for mCherry-KASH domain fusion protein under the control of the human Cd11b promoter. The GFP gRNA is specifically targeted to the GFP gene, and as a result would downregulate GFP in any infected cells in this experiment. A microglia infection rate of 75% was observed by flow cytometry (FIG. 9a). In mice injected with viruses encoding the GFP targeting gRNA, 17% of infected microglia had reduced levels of GFP (FIG. 9b). Immunohistochemistry confirmed a reduction in GFP expression in mCherry positive microglia (FIG. 9c).

### Example 9: Infection of Human Microglia Using Novel Sequences

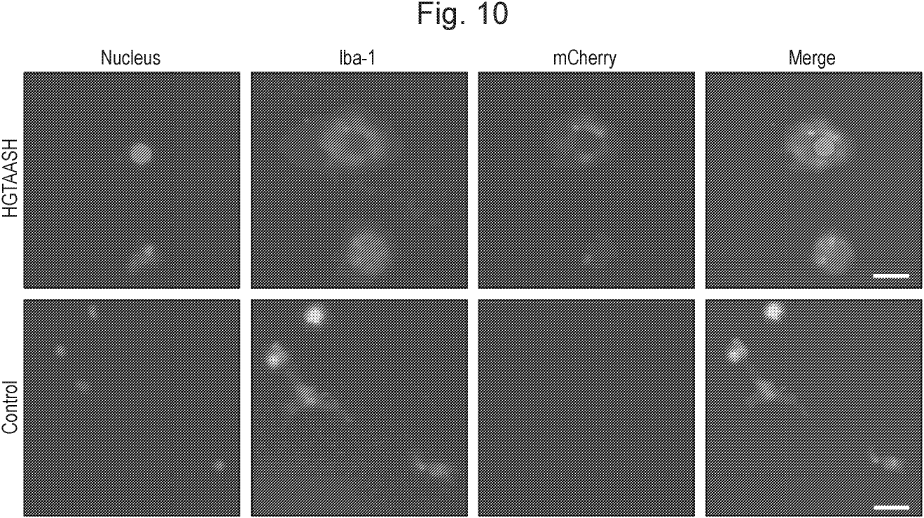

In order to determine whether the novel viruses are capable of infecting human cells, adult human microglia were isolated from a surgical biopsy. Cells were sorted using magnetic labelling for CD11b and infected with 1×10¹⁰ virus particles. Cells were cultured for 5 days and analysed for mCherry expression and expression of Iba1. In cells infected with virus encoding for mCherry under the control of a human Cd11b promoter, mCherry expression was observed in 22% of all Iba1 cells after only 5 days in culture. Conversely, microglia infected with AAV9 or PhP.EBeB serotypes did not show any significant transgene expression in Iba1 positive cells (FIG. 10). Taken together, these cells demonstrate the efficiency of infection in human microglia.

### Example 10: Novel Sequences of Microglia Infection

In order to identify binding arm sequences that can infect microglia in a comprehensive and unbiased fashion sequences were identified by DNA sequencing. As previously described, C57/B6 mice were injected with a virus containing a random screening library for novel binding arms after amino acid position 588in the VP1 capsid sequence. After a 4 week incubation, GFP positive microglia were extracted using FACS. The DNA of cells infected with a GFP transgene was prepared using lucigen DNA quick extract solution. Sequencing libraries were prepared by PCR and sequences were identified using Illumina sequencing. A list of candidate amino acid capsid sequence insertions that allow for infection of microglia is listed in Table 4. The insertion sequences provided in Table 4 may be inserted into an AAV capsid protein in the same way as described herein for any one of SEQ ID NOs: 1 to 9, or any of their variants thereof.

### Example 11: Demonstration of Binding Arm Insertion Into Additional Associated Adenoviruses

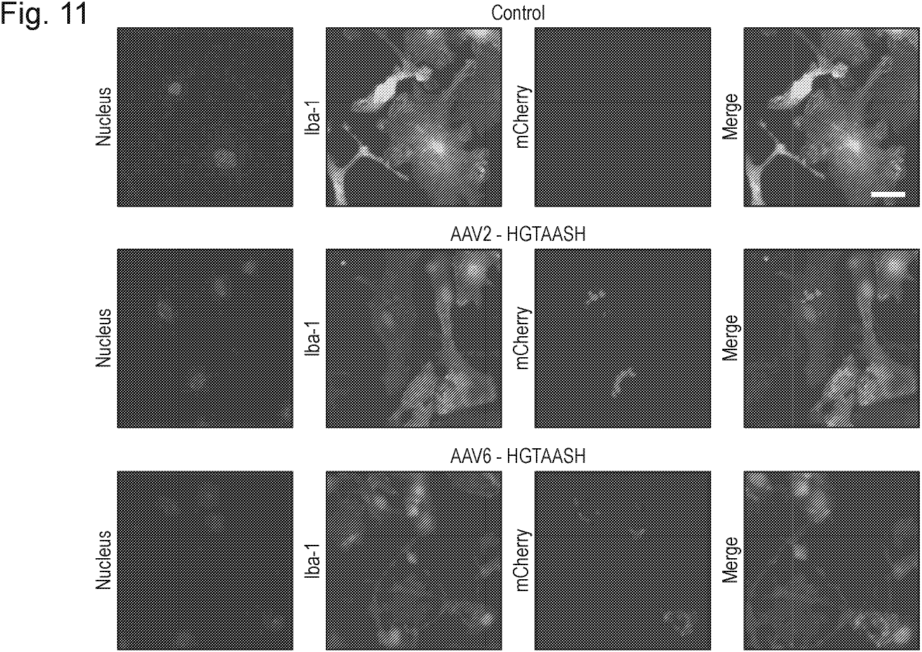

Finally to test if the insertion of the identified 7 amino acid sequences was on its own sufficient to allow for infection, the same sequence of amino acids were inserted into capsid sequences of the AAV2 and AAV6 serotypes (FIG. 11). The insertion site was identified by performing pairwise structural alignment of capsid sequences. To test if the inserted sequences would generally allow for microglia infection, the sequences were inserted into homologous regions of the AAV2 and AAV6 VP1 capsid sequences. The insertion sequences were inserted after Serine - 487 into AAV6-VP1 and after Glycine-586 into AAV2-VP1. Viruses based on these modified capsid proteins, encoding for an mCherry reporter under control of a human Cd11b promoter, were added to mouse microglia in culture. After infection 34% (AAV6-HGTAASH) and 48% (AAV2-HGTAASH) of Iba1 positive cells expressed mCherry when infected with modified capsid sequences. However, when PHP.eB capsid sequences (Control) were used, the reported low efficiency of transduction was 0%. In addition Wildtype AAV2 and AAV6 capsid viruses were tested and did not show any infection.

### INFORMAL SEQUENCE LISTING

SEQ ID NO: 1YAFGGEG

SEQ ID NO: 2ALAVPFR

SEQ ID NO: 3HGTAASH

SEQ ID NO: 4IFVMLVR

SEQ ID NO: 5LHQIPDS

SEQ ID NO: 6LYPLIDL

SEQ ID NO: 7AGTTGFP

SEQ ID NO: 8RLAEITG

SEQ ID NO: 9ALAVPFK

SEQ ID NO: 10MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GAQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWEL
QKENSKRWNPEIQYTSNYYKSNNVEFAVNTEGVYSEPRPIGTRYLTRNL

SEQ ID NO: 11MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GYAFGGEGAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 12MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GALAVPFRAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 13MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GHGTAASHAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 14MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GIFVMLVRAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 15MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GLHQIPDSAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 16MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GLYPLIDLAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 17MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GAGTTGFPAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 18MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GRLAEITGAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 19MAADGYLPDWLEDNLSEGIREWWALKPGAPQPKANQQ
HQDNARGLVLPGYKYLGPGNGLDKGEPVNAADAAALEHDKAYDQQLKAGD
NPYLKYNHADAEFQERLKEDTSFGGNLGRAVFQAKKRLLEPLGLVEEAAK
TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSD
GALAVPFKAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL

SEQ ID NO: 20ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGTATGCGTTTGGCGGAGAGGGGGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 21ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGGCTTTGGCGGTGCCTTTCAGGGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 22ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGCATGGGACAGCGGCATCGCATGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 23ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGATTTTTGTGATGCTCGTCAGGGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 24ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGTTGCATCAAATTCCTGATAGTGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 25ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGTTGTATCCTTTGATTGATCTCGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 26ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGGCTGGCACGACAGGTTTCCCGGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 27ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGCGTTTGGCGGAGATCACGGGGGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 28ATGGCTGCCGATGGTTATCTTCCAGATTGGCTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGAAAGAAGAGGCCTGT
AGAGCAGTCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACGAAGGTGCCGATGGAGTGGGTAGTTCCTCGGGAAATTGGCAT
TGCGATTCCCAATGGCTGGGGGACAGAGTCATCACCACCAGCACCCGAAC
CTGGGCCCTGCCCACCTACAACAATCACCTCTACAAGCAAATCTCCAACA
GCACATCTGGAGGATCTTCAAATGACAACGCCTACTTCGGCTACAGCACC
CCCTGGGGGTATTTTGACTTCAACAGATTCCACTGCCACTTCTCACCACG
TGACTGGCAGCGACTCATCAACAACAACTGGGGATTCCGGCCTAAGCGAC
TCAACTTCAAGCTCTTTAACATTCAGGTCAAAGAGGTTACGGACAACAAT
GGAGTCAAGACCATCGCCAATAACCTTACCAGCACGGTCCAGGTCTTCAC
GGACTCAGACTATCAGCTCCCGTACGTGCTCGGGTCGGCTCACGAGGGCT
GCCTCCCGCCGTTCCCAGCGGACGTTTTCATGATTCCTCAGTACGGGTAT
CTGACGCTTAATGATGGAAGCCAGGCCGTGGGTCGTTCGTCCTTTTACTG
CCTGGAATATTTCCCGTCGCAAATGCTAAGAACGGGTAACAACTTCCAGT
TCAGCTACGAGTTTGAGAACGTACCTTTCCATAGCAGCTACGCTCACAGC
CAAAGCCTGGACCGACTAATGAATCCACTCATCGACCAATACTTGTACTA
TCTCTCTAGAACTATTAACGGTTCTGGACAGAATCAACAAACGCTAAAAT
TCAGTGTGGCCGGACCCAGCAACATGGCTGTCCAGGGAAGAAACTACATA
CCTGGACCCAGCTACCGACAACAACGTGTCTCAACCACTGTGACTCAAAA
CAACAACAGCGAATTTGCTTGGCCTGGAGCTTCTTCTTGGGCTCTCAATG
GACGTAATAGCTTGATGAATCCTGGACCTGCTATGGCCTCTCACAAAGAA
GGAGAGGACCGTTTCTTTCCTTTGTCTGGATCTTTAATTTTTGGCAAACA
AGGTACTGGCAGAGACAACGTGGATGCGGACAAAGTCATGATAACCAACG
AAGAAGAAATTAAAACTACTAACCCGGTAGCAACGGAGTCCTATGGACAA
GTGGCCACAAACCACCAGAGTGATGGGGCTTTGGCGGTGCCTTTTAAGGC
ACAGGCGCAGACCGGTTGGGTTCAAAACCAAGGAATACTTCCGGGTATGG
TTTGGCAGGACAGAGATGTGTACCTGCAAGGACCCATTTGGGCCAAAATT
CCTCACACGGACGGCAACTTTCACCCTTCTCCGCTGATGGGAGGGTTTGG
AATGAAGCACCCGCCTCCTCAGATCCTCATCAAAAACACACCTGTACCTG
CGGATCCTCCAACGGCCTTCAACAAGGACAAGCTGAACTCTTTCATCACC
CAGTATTCTACTGGTCAAGTCAGCGTGGAGATCGAGTGGGAGCTGCAGAA
GGAAAACAGCAAGCGCTGGAACCCGGAGATCCAGTACACTTCCAACTATT
ACAAGTCTAATAATGTTGAATTTGCTGTTAATACTGAAGGTGTATATAGT
GAACCCCGCCCCATTGGCACCAGATACCTGACTCGTAATCTG

SEQ ID NO: 29TATGCGTTTGGCGGAGAGGGG

SEQ ID NO: 30GCTTTGGCGGTGCCTTTCAGG

SEQ ID NO: 31CATGGGACAGCGGCATCGCAT

SEQ ID NO: 32ATTTTTGTGATGCTCGTCAGG

SEQ ID NO: 33TTGCATCAAATTCCTGATAGT

SEQ ID NO: 34TTGTATCCTTTGATTGATCTC

SEQ ID NO: 35GCTGGCACGACAGGTTTCCCG

SEQ ID NO: 36CGTTTGGCGGAGATCACGGGG

SEQ ID NO: 37GCTTTGGCGGTGCCTTTTAAG

SEQ ID NO: 38CTAGGGGTTCCTGCGGCCTCTAGAGCCACCATGTTCA
AATTTGAACTGACTAAGCGGCTC

SEQ ID NO: 39AGCGGTCGCAGAGGAGCG

SEQ ID NO: 40TCCCGCTCCTCTGCGACCGCTATGGCTGCCGATGGTT
ATCT

SEQ ID NO: 41CAGATTACGAGTCAGGTATCTGGTG

SEQ ID NO: 42GATACCTGACTCGTAATCTGGAGGGCAGAGGAAGTCT
GCTAACATGCGG

SEQ ID NO: 43ACTAGGGGTTCCTGCGGCCGCACACAAAAAACCAACA
CACAGATCTAATGGCTCCGGGTAT

SEQ ID NO: 44GTCGACTTGCTTGTTAATCAATAAACCG

SEQ ID NO: 45CCCATCACTCTGGTGGTTTGTG

SEQ ID NO: 46TTGATGAATCCTGGACCTG

SEQ ID NO: 47AGTTCAGCTTGTCCTTGTTG

SEQ ID NO: 48GTATTCCTTGGTTTTGAACCCAACCGGTCTGCGCCTG
TGCNNNNNNNNNNNNNNNNNNNNNCCCATCACTCTGGTGGTTTGTGGCCA
CTTGTCCATAGGA

SEQ ID NO: 49ATGGCTGCCGATGGTTGACTTCCAGATTAACTCGAGG
ACAACCTTAGTGAAGGAATTCGCGAGTGGTGGGCTTTGAAACCTGGAGCC
CCTCAACCCAAGGCAAATCAACAACATCAAGACAACGCTAGAGGTCTTGT
GCTTCCGGGTTACAAATACCTTGGACCCGGCAACGGACTCGACAAGGGGG
AGCCGGTCAACGCAGCAGACGCGGCGGCCCTCGAGCACGACAAAGCCTAC
GACCAGCAGCTCAAGGCCGGAGACAACCCGTACCTCAAGTACAACCACGC
CGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTT
GGTCTGGTTGAGGAAGCGGCTAAGACGGCTCCTGGATGAAAGAGGCCTGT
AGAGTGATCTCCTCAGGAACCGGACTCCTCCGCGGGTATTGGCAAATCGG
GTGCACAGCCCGCTAAAAAGAGACTCAATTTCGGTCAGACTGGCGACACA
GAGTCAGTCCCAGACCCTCAACCAATCGGAGAACCTCCCGCAGCCCCCTC
AGGTGTGGGATCTCTTACAATGGCTTCAGGTGGTGGCGCACCAGTGGCAG
ACAATAACTGAGGTGCCGATGGAGTGGGTAGTTCC

SEQ ID NO: 50TCCTATGGACAAGTGGCCACAAACCACCAGAGTGATG
GGGCTTTGGCGGTGCCTTTTAAGGCACAGGCGCAGACCGGTTGGGTTCAA
AACCAAGGAATAC

SEQ ID NO: 51TCCTATGGACAAGTGGCCACAAACCACCAGAGTGATG
GGGCTTTGGCGGTGCCTTTCAGGGCACAGGCGCAGACCGGTTGGGTTCAA
AACCAAGGAATAC

SEQ ID NO: 52TCCTATGGACAAGTGGCCACAAACCACCAGAGTGATG
GGCATGGGACAGCGGCATCGCATGCACAGGCGCAGACCGGTTGGGTTCAA
AACCAAGGAATAC

SEQ ID NO: 53TCCTATGGACAAGTGGCCACAAACCACCAGAGTGATG
GGTATGCGTTTGGCGGAGAGGGGGCACAGGCGCAGACCGGTTGGGTTCAA
AACCAAGGAATAC

SEQ ID NO: 362MAADGYLPDWLEDNLSEGIREWWDLKPGAPKPKANQ
QKQDDGRGLVLPGYKYLGPFNGLDKGEPVNAADAAALEHDKAYDQQLKAG
DNPYLRYNHADAEFQERLQEDTSFGGNLGRAVFQAKKRVLEPFGLVEEGA
KTAPGKKRPVEQSPQEPDSSSGIGKTGQQPAKKRLNFGQTGDSESVPDPQ
PLGEPPATPAAVGPTTMASGGGAPMADNNEGADGVGNASGNWHCDSTWLG
DRVITTSTRTWALPTYNNHLYKQISSASTGASNDNHYFGYSTPWGYFDFN
RFHCHFSPRDWQRLINNNWGFRPKRLNFKLFNIQVKEVTTNDGVTTIANN
LTSTVQVFSDSEYQLPYVLGSAHQGCLPPFPADVFMIPQYGYLTLNNGSQ
AVGRSSFYCLEYFPSQMLRTGNNFTFSYTFEDVPFHSSYAHSQSLDRLMN
PLIDQYLYYLNRTQNQSGSAQNKDLLFSRGSPAGMSVQPKNWLPGPCYRQ
QRVSKTKTDNNNSNFTWTGASKYNLNGRESIINPGTAMASHKDDKDKFFP
MSGVMIFGKESAGASNTALDNVMITDEEEIKATNPVATERFGTVAVNLQS
SSTDPATGDVHVMGALPGMVWQDRDVYLQGPIWAKIPHTDGHFHPSPLMG
GFGLKHPPPQILIKNTPVPANPPAEFSATKFASFITQYSTGQVSVEIEWE
LQKENSKRWNPEVQYTSNYAKSANVDFTVDNNGLYTEPRPIGTRYLTRPL

SEQ ID NO: 363MAADGYLPDWLEDTLSEGIRQWWKLKPGPPPPKPAE
RHKDDSRGLVLPGYKYLGPFNGLDKGEPVNEADAAALEHDKAYDRQLDSG
DNPYLKYNHADAEFQERLKEDTSFGGNLGRAVFQAKKRVLEPLGLVEEPV
KTAPGKKRPVEHSPVEPDSSSGTGKAGQQPARKRLNFGQTGDADSVPDPQ
PLGQPPAAPSGLGTNTMATGSGAPMADNNEGADGVGNSSGNWHCDSTWMG
DRVITTSTRTWALPTYNNHLYKQISSQSGASNDNHYFGYSTPWGYFDFNR
FHCHFSPRDWQRLINNNWGFRPKRLNFKLFNIQVKEVTQNDGTTTIANNL
TSTVQVFTDSEYQLPYVLGSAHQGCLPPFPADVFMVPQYGYLTLNNGSQA
VGRSSFYCLEYFPSQMLRTGNNFTFSYTFEDVPFHSSYAHSQSLDRLMNP
LIDQYLYYLSRTNTPSGTTTQSRLQFSQAGASDIRDQSRNWLPGPCYRQQ
RVSKTSADNNNSEYSWTGATKYHLNGRDSLVNPGPAMASHKDDEEKFFPQ
SGVLIFGKQGSEKTNVDIEKVMITDEEEIRTTNPVATEQYGSVSTNLQRG
NRQAATADVNTQGVLPGMVWQDRDVYLQGPIWAKIPHTDGHFHPSPLMGG
FGLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRNL

SEQ ID NO: 364MAADGYLPDWLEDNLSEGIREWWDLKPGAPKPKANQ
QKQDDGRGLVLPGYKYLGPFNGLDKGEPVNAADAAALEHDKAYDQQLKAG
DNPYLRYNHADAEFQERLQEDTSFGGNLGRAVFQAKKRVLEPFGLVEEGA
KTAPGKKRPVEQSPQEPDSSSGIGKTGQQPAKKRLNFGQTGDSESVPDPQ
PLGEPPATPAAVGPTTMASGGGAPMADNNEGADGVGNASGNWHCDSTWLG
DRVITTSTRTWALPTYNNHLYKQISSASTGASNDNHYFGYSTPWGYFDFN
RFHCHFSPRDWQRLINNNWGFRPKRLNFKLFNIQVKEVTTNDGVTTIANN
LTSTVQVFSDSEYQLPYVLGSAHQGCLPPFPADVFMIPQYGYLTLNNGSQ
AVGRSSFYCLEYFPSQMLRTGNNFTFSYTFEDVPFHSSYAHSQSLDRLMN
PLIDQYLYYLNRTQNQSGSAQNKDLLFSRGSPAGMSVQPKNWLPGPCYRQ
QRVSKTKTDNNNSNFTWTGASKYNLNGRESIINPGTAMASHKDDKDKFFP
MSGVMIFGKESAGASNTALDNVMITDEEEIKATNPVATERFGTVAVNLQS
SHGTAASHSTDPATGDVHVMGALPGMVWQDRDVYLQGPIWAKIPHTDGHF
HPSPLMGGFGLKHPPPQILIKNTPVPANPPAEFSATKFASFITQYSTGQV
SVEIEWELQKENSKRWNPEVQYTSNYAKSANVDFTVDNNGLYTEPRPIGT
RYLTRPL

SEQ ID NO: 365MAADGYLPDWLEDTLSEGIRQWWKLKPGPPPPKPAE
RHKDDSRGLVLPGYKYLGPFNGLDKGEPVNEADAAALEHDKAYDRQLDSG
DNPYLKYNHADAEFQERLKEDTSFGGNLGRAVFQAKKRVLEPLGLVEEPV
KTAPGKKRPVEHSPVEPDSSSGTGKAGQQPARKRLNFGQTGDADSVPDPQ
PLGQPPAAPSGLGTNTMATGSGAPMADNNEGADGVGNSSGNWHCDSTWMG
DRVITTSTRTWALPTYNNHLYKQISSQSGASNDNHYFGYSTPWGYFDFNR
FHCHFSPRDWQRLINNNWGFRPKRLNFKLFNIQVKEVTQNDGTTTIANNL
TSTVQVFTDSEYQLPYVLGSAHQGCLPPFPADVFMVPQYGYLTLNNGSQA
VGRSSFYCLEYFPSQMLRTGNNFTFSYTFEDVPFHSSYAHSQSLDRLMNP
LIDQYLYYLSRTNTPSGTTTQSRLQFSQAGASDIRDQSRNWLPGPCYRQQ
RVSKTSADNNNSEYSWTGATKYHLNGRDSLVNPGPAMASHKDDEEKFFPQ
SGVLIFGKQGSEKTNVDIEKVMITDEEEIRTTNPVATEQYGSVSTNLQRG
HGTAASHNRQAATADVNTQGVLPGMVWQDRDVYLQGPIWAKIPHTDGHFH
PSPLMGGFGLKHPPPQILIKNTPVPANPSTTFSAAKFASFITQYSTGQVS
VEIEWELQKENSKRWNPEIQYTSNYNKSVNVDFTVDTNGVYSEPRPIGTR
YLTRNL

### LIST OF TABLES

TABLE 1

| | | |
|---|---|---|
| Gene | Forward | Reverse |
| AAV replication | 5′-ctaggggttcctgcggcctctagaGCCAccatgttcaaatttgaactgactaagcggctc-3′ | 5′-agcggtcgcagaggagcg-3′ |
| Capsid protein (3′) | 5′-tcccgctcctctgcgaccgctatggctgccgatggttatct-3′ | 5′-cagattacgagtcaggtatctggtg-3′ |
| T2A-GFP | 5′-gatacctgactcgtaatctggagggcagaggaagtctgctaacatgcgg-3′ | 5′-actaggggttcctgcggccgcacacaaaaaaccaacacacagatctaatggctccgggtat-3′ |
| PHP.e8 | 5′-gtcgacttgcttgttaatcaataaaccg-3′ | 5′-cccatcactctggtggtttgtg-3′ |
| Binding arm | 5′-ttgatgaatcctggacctg-3′ | 5′-agttcagcttgtccttgttg-3′ |

TABLE 2

| | |
|---|---|
| Fragment | Sequence |
| Random library | 5′-gtattccttggttttgaacccaaccggtctgcgcctgtgcNNNNNNNNNNNNNNNNNNNNNcccatcactctggtggtttgtggccacttgtccatagga-3′ |
| Cap Gene with stop codon insertions | 5′-atggctgccgatggttGActtccagattAActcgaggacaaccttagtgaaggaattcgcgagtggtgggctttgaaacctggagcccctcaacccaaggcaaatcaacaacatcaagacaacg ctagaggtcttgtgcttccgggttacaaataccttggacccggcaacggactcgacaagggggagccggtcaacgcagcagacgcggcggccctcgagcacgacaaagcctacgaccagcagct caaggccggagacaacccgtacctcaagtacaaccacgccgacgccgagttccaggagcggctcaaagaagatacgtcttttgggggcaacctcgggcgagtcttccaggccaaaaagag gcttcttgaacctcttggtctggttgaggaagcggctaagacggctcctggaTGaaagaggcctgtagagTGAtctcctcaggaaccggactcctccgcgggtattggcaaatcgggtgcatagc ccactaaaaagagactcaatttcggtcagactggcgacacagagtcagtcccagaccctcaaccaatcggagaacctcccgcagccccctcaggtgtgggatctcttacaatggcttcaggtggtg gcgcaccagtggcagacaataacTGAggtgccgatggagtgggtagttcc-3′ |
| Novel sequence 1 | 5′-tcctatggacaagtggccacaaaccaccagagtgatggggctttggcggtgccttttaaggcacaggcgcagaccggttgggttcaaaaccaaggaatac-3 |
| Novel sequence 2 | 5′-tcctatggacaagtggccacaaaccaccagagtgatggggctttggcggtgcctttcagggcacaggcgcagaccggttgggttcaaaaccaaggaatac-3′ |
| Novel sequence 3 | 5′-tcctatggacaagtggccacaaaccaccagagtgatgggcatgggacacagcggcatcgcacaggcgcagaccggttgggttcaaaaccaaggaatac-3′ |
| Novel sequence 4 | 5′-tcctatggacaagtggccacaaaccaccagagtgatgggtatgcgtttggcggagagggggcacaggcgcagaccggttgggttcaaaaccaaggaatac-3′ |

TABLE 3

IFVMLVR
LHQIPDS^{∗}
LYPLIDL
AGTTGFP
RLAEITG

TABLE 4

| | | | |
|---|---|---|---|
| SEQ ID NO (amino acid) | Amino acid sequence | SEQ ID NO (DNA) | DNA sequence |
| SEQ ID NO: 54 | IFFLNVT | SEQ ID NO: 208 | ATTTTTTTTTTGAATGTTACG |
| SEQ ID NO: 55 | IFFLNDT | SEQ ID NO: 209 | ATTTTTTTTTTGAATGATACG |
| SEQ ID NO: 56 | IFFFNVT | SEQ ID NO: 210 | ATTTTTTTTTTTAATGTTACG |
| SEQ ID NO: 57 | SFFLNYT | SEQ ID NO: 211 | TCTTTTTTTTTAAATTATACG |
| SEQ ID NO: 58 | IFFFNDT | SEQ ID NO: 212 | ATTTTTTTTTTTAATGATACG |
| SEQ ID NO: 59 | SFFFNDT | SEQ ID NO: 213 | TCTTTTTTTTTTAATGATACG |
| SEQ ID NO: 60 | IFFLNYT | SEQ ID NO: 214 | ATTTTTTTTTTGAATTATACG |
| SEQ ID NO: 61 | SFFFNYT | SEQ ID NO: 215 | TCTTTTTTTTTTAATTATACG |
| SEQ ID NO: 62 | SFFLNDT | SEQ ID NO: 216 | TCTTTTTTTTTGAATGATACG |
| SEQ ID NO: 63 | IFFFNYT | SEQ ID NO: 217 | ATTTTTTTTTTTAATTATACG |
| SEQ ID NO: 64 | SFFLNVT | SEQ ID NO: 218 | TCTTTTTTTTTGAATGTTACG |
| SEQ ID NO: 65 | FFFLNVT | SEQ ID NO: 219 | TTTTTTTTTTTGAATGTTACG |
| SEQ ID NO: 66 | SFFLTVT | SEQ ID NO: 220 | TCTTTTTTTTTGACTGTTACG |
| SEQ ID NO: 67 | FFFFNYT | SEQ ID NO: 221 | TTTTTTTTTTTTAATTATACG |
| SEQ ID NO: 68 | FFFFNDT | SEQ ID NO: 222 | TTTTTTTTTTTTAATGATACG |
| SEQ ID NO: 69 | SFFFNVT | SEQ ID NO: 223 | TCTTTTTTTTTTAATGTTACG |
| SEQ ID NO: 70 | FFFFNVT | SEQ ID NO: 224 | TTTTTTTTTTTTAATGTTACG |
| SEQ ID NO: 71 | FFVLNVT | SEQ ID NO: 225 | TTTTTTGTTTTGAATGTTACG |
| SEQ ID NO: 72 | PPPPRPP | SEQ ID NO: 226 | CCCCCCCCCCCGCGACCCCCG |
| SEQ ID NO: 73 | NSNSHSF | SEQ ID NO: 227 | AACAGCAATTCACACTCTTTC |
| SEQ ID NO: 74 | IFLFNYT | SEQ ID NO: 228 | ATTTTTTTGTTTAATTATACG |
| SEQ ID NO: 75 | FFFLNDT | SEQ ID NO: 229 | TTTTTTTTTTTGAATGATACG |
| SEQ ID NO: 76 | SFVFNIT | SEQ ID NO: 230 | TCTTTTGTTTTTAATATTACG |
| SEQ ID NO: 77 | FFFLNYT | SEQ ID NO: 231 | TTTTTTTTTTTGAATTATACG |
| SEQ ID NO: 78 | IFFLTIR | SEQ ID NO: 232 | ATTTTTTTTTTAACGATCCGG |
| SEQ ID NO: 79 | NRNLHSN | SEQ ID NO: 233 | AATAGGAATTTACACTCTAAC |
| SEQ ID NO: 80 | SFLLNYT | SEQ ID NO: 234 | TCTTTTTTGTTGAATTATACG |
| SEQ ID NO: 81 | FFFLTDQ | SEQ ID NO: 235 | TTTTTTTTTTTAACTGATCAG |
| SEQ ID NO: 82 | SFFFNCT | SEQ ID NO: 236 | TCTTTTTTTTTTAATTGTACG |
| SEQ ID NO: 83 | IFVLNYT | SEQ ID NO: 237 | ATTTTTGTTTTGAATTATACG |
| SEQ ID NO: 84 | SFFFTVT | SEQ ID NO: 238 | TCTTTTTTTTTTACTGTTACG |
| SEQ ID NO: 85 | SFSLNYT | SEQ ID NO: 239 | TCTTTTTCTTTAAATTATACG |
| SEQ ID NO: 86 | IFVFNYT | SEQ ID NO: 240 | ATTTTTGTTTTTAATTATACG |
| SEQ ID NO: 87 | IFFFTYT | SEQ ID NO: 241 | ATTTTTTTTTTTACGTATACG |
| SEQ ID NO: 88 | SFLFNYT | SEQ ID NO: 242 | TCTTTTTTGTTTAATTATACG |
| SEQ ID NO: 89 | FFFFTDT | SEQ ID NO: 243 | TTTTTTTTTTTTACTGATACG |
| SEQ ID NO: 90 | IFFLTYT | SEQ ID NO: 244 | ATTTTTTTTTTGACTTATACG |
| SEQ ID NO: 91 | SFVLTVQ | SEQ ID NO: 245 | TCTTTTGTTTTGACTGTTCAG |
| SEQ ID NO: 92 | SFSFTYT | SEQ ID NO: 246 | TCTTTTTCTTTTACTTATACG |
| SEQ ID NO: 93 | NFFLNYT | SEQ ID NO: 247 | AATTTTTTTTTGAATTATACG |
| SEQ ID NO: 94 | SFSLNVT | SEQ ID NO: 248 | TCTTTTTCTTTGAATGTTACG |
| SEQ ID NO: 95 | FFLFNVT | SEQ ID NO: 249 | TTTTTTTTGTTTAATGTTACG |
| SEQ ID NO: 96 | FFVLIDT | SEQ ID NO: 250 | TTTTTTGTGTTGATTGATACG |
| SEQ ID NO: 97 | IFVLNDT | SEQ ID NO: 251 | ATTTTTGTTTTAAATGATACG |
| SEQ ID NO: 98 | AFVLNYT | SEQ ID NO: 252 | GCTTTTGTTTTGAATTATACG |
| SEQ ID NO: 99 | FFLLNVT | SEQ ID NO: 253 | TTTTTTTTGTTAAATGTTACG |
| SEQ ID NO: 100 | SFFLNCT | SEQ ID NO: 254 | TCTTTTTTTTTAAATTGTACG |
| SEQ ID NO: 101 | IFFFNVP | SEQ ID NO: 255 | ATTTTTTTTTTTAATGTTCCG |
| SEQ ID NO: 102 | LFFLNYT | SEQ ID NO: 256 | CTTTTTTTTTTAAATTATACG |
| SEQ ID NO: 103 | FFVKHIT | SEQ ID NO: 257 | TTTTTTGTGAAGCATATTACG |
| SEQ ID NO: 104 | IFSKRYR | SEQ ID NO: 258 | ATTTTTTCTAAACGATACAGG |
| SEQ ID NO: 105 | FFFLTVT | SEQ ID NO: 259 | TTTTTTTTTTTAACTGTTACG |
| SEQ ID NO: 106 | IFFFTVT | SEQ ID NO: 260 | ATTTTTTTTTTTACTGTTACG |
| SEQ ID NO: 107 | SFFFTDT | SEQ ID NO: 261 | TCTTTTTTTTTTACGGATACG |
| SEQ ID NO: 108 | FFFLTDT | SEQ ID NO: 262 | TTTTTTTTTTTGACTGATACG |
| SEQ ID NO: 109 | IFFLTDT | SEQ ID NO: 263 | ATTTTTTTTTTAACTGATACG |
| SEQ ID NO: 110 | AFFLTYT | SEQ ID NO: 264 | GCTTTTTTTTTGACTTATACG |
| SEQ ID NO: 111 | NFFLTYP | SEQ ID NO: 265 | AATTTTTTTTTGACTTATCCG |
| SEQ ID NO: 112 | IFFFSYT | SEQ ID NO: 266 | ATTTTTTTTTTTAGTTATACG |
| SEQ ID NO: 113 | IFLFNVT | SEQ ID NO: 267 | ATTTTTTTGTTTAATGTTACG |
| SEQ ID NO: 114 | PRDLHSN | SEQ ID NO: 268 | CCCCGAGATCTACACAGCAAT |
| SEQ ID NO: 115 | SFLLNVT | SEQ ID NO: 269 | TCTTTTTTGTTGAATGTTACG |
| SEQ ID NO: 116 | HSNSHSF | SEQ ID NO: 270 | CACAGCAATTCACACTCTTTC |
| SEQ ID NO: 117 | HRDLHSN | SEQ ID NO: 271 | CACCGAGATCTACACAGCAAT |
| SEQ ID NO: 118 | NFLLNYT | SEQ ID NO: 272 | AATTTTTTGTTGAATTATACG |
| SEQ ID NO: 119 | IFVFNDT | SEQ ID NO: 273 | ATTTTTGTTTTTAATGATACG |
| SEQ ID NO: 120 | IFFLTVT | SEQ ID NO: 274 | ATTTTTTTTTTGACTGTTACG |
| SEQ ID NO: 121 | FFFLILR | SEQ ID NO: 275 | TTTTTTTTTTTAATTTTACGG |
| SEQ ID NO: 122 | IWGLLLR | SEQ ID NO: 276 | ATTTGGGGGTTGCTTTTAAGG |
| SEQ ID NO: 123 | FFFLTLR | SEQ ID NO: 277 | TTTTTTTTTTTAACTTTAAGG |
| SEQ ID NO: 124 | IFFLMIR | SEQ ID NO: 278 | ATTTTTTTTTTAATGATCCGG |
| SEQ ID NO: 125 | HMKAHSN | SEQ ID NO: 279 | CACATGAAGGCGCACTCTAAC |
| SEQ ID NO: 126 | HRNLHSY | SEQ ID NO: 280 | CACAGGAATTTACACTCTTAC |
| SEQ ID NO: 127 | FFFKDTT | SEQ ID NO: 281 | TTTTTTTTTAAGGATACGACG |
| SEQ ID NO: 128 | SFVLNYT | SEQ ID NO: 282 | TCTTTTGTTTTGAATTATACG |
| SEQ ID NO: 129 | FFFKNPT | SEQ ID NO: 283 | TTTTTTTTTAAGAATCCGACG |
| SEQ ID NO: 130 | NYRDLHS | SEQ ID NO: 284 | AACTACCGAGATCTACACAGC |
| SEQ ID NO: 131 | FFLKHLA | SEQ ID NO: 285 | TTTTTTTTGAAGCATCTGGCG |
| SEQ ID NO: 132 | PWELHSN | SEQ ID NO: 286 | CCCTGGGAGCTGCACAGCAAT |
| SEQ ID NO: 133 | IFFFTDT | SEQ ID NO: 287 | ATTTTTTTTTTTACTGATACG |
| SEQ ID NO: 134 | SFVFNVT | SEQ ID NO: 288 | TCTTTTGTTTTTAATGTTACG |
| SEQ ID NO: 135 | PFFLTYT | SEQ ID NO: 289 | CCTTTTTTTTTAACTTATACG |
| SEQ ID NO: 136 | FFVLNDT | SEQ ID NO: 290 | TTTTTTGTTTTGAATGATACG |
| SEQ ID NO: 137 | YFFLNDT | SEQ ID NO: 291 | TATTTTTTTTTGAATGATACG |
| SEQ ID NO: 138 | SFLFNDT | SEQ ID NO: 292 | TCTTTTTTGTTTAATGATACG |
| SEQ ID NO: 139 | IFFERCG | SEQ ID NO: 293 | ATTTTTTTTGAGAGATGCGGG |
| SEQ ID NO: 140 | NRELHSN | SEQ ID NO: 294 | AACCGAGAGCTACACAGCAAT |
| SEQ ID NO: 141 | NFVFHYT | SEQ ID NO: 295 | AATTTTGTGTTTCATTATACG |
| SEQ ID NO: 142 | AFGELHN | SEQ ID NO: 296 | GCCTTCGGGGAGCTACACAAC |
| SEQ ID NO: 143 | IFFESSR | SEQ ID NO: 297 | ATTTTTTTTGAGTCATCTAGG |
| SEQ ID NO: 144 | PRDLHIN | SEQ ID NO: 298 | CCCCGAGATCTACACATCAAT |
| SEQ ID NO: 145 | IFLLHVT | SEQ ID NO: 299 | ATTTTTTTGTTGCATGTTACG |
| SEQ ID NO: 146 | NRNSHSI | SEQ ID NO: 300 | AACAGGAATTCACACTCTATC |
| SEQ ID NO: 147 | HRTAASH | SEQ ID NO: 301 | CATCGGACAGCGGCATCGCAT |
| SEQ ID NO: 148 | NGTAASH | SEQ ID NO: 302 | AATGGGACAGCGGCATCGCAT |
| SEQ ID NO: 149 | HGTAASQ | SEQ ID NO: 303 | CATGGGACAGCGGCATCGCAG |
| SEQ ID NO: 150 | HGAAASH | SEQ ID NO: 304 | CATGGGGCAGCGGCATCGCAT |
| SEQ ID NO: 151 | HGTAALN | SEQ ID NO: 305 | CATGGGACAGCGGCATTGAAT |
| SEQ ID NO: 152 | NGTAASQ | SEQ ID NO: 306 | AATGGGACAGCGGCATCGCAG |
| SEQ ID NO: 153 | NGTAASN | SEQ ID NO: 307 | AATGGGACAGCGGCATCGAAT |
| SEQ ID NO: 154 | HLTVASQ | SEQ ID NO: 308 | CATTTGACAGTGGCATCTCAG |
| SEQ ID NO: 155 | IFVLNVT | SEQ ID NO: 309 | ATTTTTGTTTTGAATGTTACG |
| SEQ ID NO: 156 | IFVFNVT | SEQ ID NO: 310 | ATTTTTGTTTTTAATGTTACG |
| SEQ ID NO: 157 | IFLLMLR | SEQ ID NO: 311 | ATTTTTTTGTTAATGTTACGG |
| SEQ ID NO: 158 | IFVLNVK | SEQ ID NO: 312 | ATTTTTGTTTTGAATGTTAAG |
| SEQ ID NO: 159 | IFVVNVT | SEQ ID NO: 313 | ATTTTTGTTGTGAATGTTACG |
| SEQ ID NO: 160 | IFFVMIR | SEQ ID NO: 314 | ATTTTTTTTGTAATGATACGG |
| SEQ ID NO: 161 | IFVVPSR | SEQ ID NO: 315 | ATTTTTGTGGTGCCTTCTAGG |
| SEQ ID NO: 162 | ALAVAFK | SEQ ID NO: 316 | GCTTTGGCGGTGGCTTTTAAG |
| SEQ ID NO: 163 | TLAVPFR | SEQ ID NO: 317 | ACTTTGGCGGTGCCTTTTAGG |
| SEQ ID NO: 164 | ILAVPFR | SEQ ID NO: 318 | ATTTTGGCGGTGCCTTTTAGG |
| SEQ ID NO: 165 | TLAVPFN | SEQ ID NO: 319 | ACTTTGGCGGTGCCTTTTAAT |
| SEQ ID NO: 166 | TLAVPFE | SEQ ID NO: 320 | ACTTTGGCGGTGCCTTTTGAG |
| SEQ ID NO: 167 | TLAVPFT | SEQ ID NO: 321 | ACTTTGGCGGTGCCTTTTACG |
| SEQ ID NO: 168 | TLAVPFQ | SEQ ID NO: 322 | ACTTTGGCGGTGCCTTTTCAG |
| SEQ ID NO: 169 | TLAVPFM | SEQ ID NO: 323 | ACTTTGGCGGTGCCTTTTATG |
| SEQ ID NO: 170 | TLALPFK | SEQ ID NO: 324 | ACTTTGGCGTTGCCTTTTAAG |
| SEQ ID NO: 171 | TLAVLFK | SEQ ID NO: 325 | ACTTTGGCGGTGCTTTTTAAG |
| SEQ ID NO: 172 | TLAVPSK | SEQ ID NO: 326 | ACTTTGGCGGTGCCTTCTAAG |
| SEQ ID NO: 173 | TSAVPFK | SEQ ID NO: 327 | ACTTCGGCGGTGCCTTTTAAG |
| SEQ ID NO: 174 | TLTVPFK | SEQ ID NO: 328 | ACTTTGACGGTGCCTTTTAAG |
| SEQ ID NO: 175 | NLAGPFK | SEQ ID NO: 329 | AACTTGGCGGGTCCCTTTAAG |
| SEQ ID NO: 176 | NLAVPFK | SEQ ID NO: 330 | AACTTGGCGGTGCCTTTTAAG |
| SEQ ID NO: 177 | ILAVPFK | SEQ ID NO: 331 | ATTTTGGCGGTGCCTTTTAAG |
| SEQ ID NO: 178 | PLAVPFK | SEQ ID NO: 332 | CCTTTGGCGGTGCCTTTTAAG |
| SEQ ID NO: 179 | SLAVPFK | SEQ ID NO: 333 | TCTTTGGCGGTGCCTTTTAAG |
| SEQ ID NO: 180 | DLAVPFK | SEQ ID NO: 334 | GATTTGGCGGTGCCTTTTAAG |
| SEQ ID NO: 181 | ALAGPFK | SEQ ID NO: 335 | GCTTTGGCGGGGCCTTTTAAG |
| SEQ ID NO: 182 | TLAGPFK | SEQ ID NO: 336 | ACTTTGGCGGGGCCTTTTAAG |
| SEQ ID NO: 183 | TWAVPFK | SEQ ID NO: 337 | ACTTGGGCGGTGCCTTTTAAG |
| SEQ ID NO: 184 | TFAVPFK | SEQ ID NO: 338 | ACTTTTGCGGTGCCTTTTAAG |
| SEQ ID NO: 185 | TLAVHFK | SEQ ID NO: 339 | ACTTTGGCGGTGCATTTTAAG |
| SEQ ID NO: 186 | TLAVPLK | SEQ ID NO: 340 | ACTTTGGCGGTGCCTTTAAAG |
| SEQ ID NO: 187 | TLAVTFK | SEQ ID NO: 341 | ACTTTGGCGGTGACTTTTAAG |
| SEQ ID NO: 188 | TVAVPFK | SEQ ID NO: 342 | ACTGTGGCGGTGCCTTTTAAG |
| SEQ ID NO: 189 | TLVVPFK | SEQ ID NO: 343 | ACTTTGGTGGTGCCTTTTAAG |
| SEQ ID NO: 190 | TLAVPCK | SEQ ID NO: 344 | ACTTTGGCGGTGCCTTGTAAG |
| SEQ ID NO: 191 | TLEVPFK | SEQ ID NO: 345 | ACTTTGGAGGTGCCTTTTAAG |
| SEQ ID NO: 192 | TLAAPFK | SEQ ID NO: 346 | ACTTTGGCGGCGCCTTTTAAG |
| SEQ ID NO: 193 | TLSVPFK | SEQ ID NO: 347 | ACTTTGTCGGTGCCTTTTAAG |
| SEQ ID NO: 194 | TLAVRFK | SEQ ID NO: 348 | ACTTTGGCGGTGCGTTTTAAG |
| SEQ ID NO: 195 | TLGVPFK | SEQ ID NO: 349 | ACTTTGGGGGTGCCTTTTAAG |
| SEQ ID NO: 196 | TLAVPVK | SEQ ID NO: 350 | ACTTTGGCGGTGCCTGTTAAG |
| SEQ ID NO: 197 | TLAVAFK | SEQ ID NO: 351 | ACTTTGGCGGTGGCTTTTAAG |
| SEQ ID NO: 198 | TLAVSFK | SEQ ID NO: 352 | ACTTTGGCGGTGTCTTTTAAG |
| SEQ ID NO: 199 | TLAMPFK | SEQ ID NO: 353 | ACTTTGGCGATGCCTTTTAAG |
| SEQ ID NO: 200 | TMAVPFK | SEQ ID NO: 354 | ACTATGGCGGTGCCTTTTAAG |
| SEQ ID NO: 201 | TLAVPYK | SEQ ID NO: 355 | ACTTTGGCGGTGCCTTATAAG |
| SEQ ID NO: 202 | TLAEPFK | SEQ ID NO: 356 | ACTTTGGCGGAGCCTTTTAAG |
| SEQ ID NO: 203 | TLAVPIK | SEQ ID NO: 357 | ACTTTGGCGGTGCCTATTAAG |
| SEQ ID NO: 204 | TFAGPFK | SEQ ID NO: 358 | ACTTTTGCGGGGCCTTTTAAG |
| SEQ ID NO: 205 | TLAGPLK | SEQ ID NO: 359 | ACTTTGGCGGGGCCTTTAAAG |
| SEQ ID NO: 206 | NLGGPFK | SEQ ID NO: 360 | AACTTGGGGGGGCCTTTTTAAG |
| SEQ ID NO: 207 | TLAGAFK | SEQ ID NO: 361 | ACTTTGGCCGGGGCCTTTAAG |

1. An adeno-associated virus (AAV) capsid protein, wherein the AAV capsid protein has been modified to insert an amino acid sequence having:
(a) the amino acid sequence of SEQ ID NO: 1, or a variant thereof having a single amino acid substitution;
(b) the amino acid sequence of SEQ ID NO: 2, or a variant thereof having a single amino acid substitution;
(c) the amino acid sequence of SEQ ID NO: 3, or a variant thereof having a single amino acid substitution;
(d) the amino acid sequence of SEQ ID NO: 4, or a variant thereof having a single amino acid substitution;
(e) the amino acid sequence of SEQ ID NO: 5, or a variant thereof having a single amino acid substitution;
(f) the amino acid sequence of SEQ ID NO: 6, or a variant thereof having a single amino acid substitution;
(g) the amino acid sequence of SEQ ID NO: 7, or a variant thereof having a single amino acid substitution;
(h) the amino acid sequence of SEQ ID NO: 8, or a variant thereof having a single amino acid substitution; or
(i) the amino acid sequence of SEQ ID NO: 9.

2. The AAV capsid protein of claim 1, wherein the amino acid sequence insertion results in increased transduction efficiency in microglia or brain macrophages compared to an AAV capsid protein without the amino acid insertion.

3. The AAV capsid protein of claim 1 or 2, wherein the AAV capsid protein has been modified to insert an amino acid sequence having:
(a) the amino acid sequence of SEQ ID NO: 1;
(b) the amino acid sequence of SEQ ID NO: 2;
(c) the amino acid sequence of SEQ ID NO: 3;
(d) the amino acid sequence of SEQ ID NO: 4;
(e) the amino acid sequence of SEQ ID NO: 5;
(f) the amino acid sequence of SEQ ID NO: 6;
(g) the amino acid sequence of SEQ ID NO: 7; or
(h) the amino acid sequence of SEQ ID NO: 8.

4. The AAV capsid protein of any one of claims 1 to 3, wherein the unmodified AAV capsid protein is a wild-type AAV1, AAV2, AAV5, AAV7, AAV8, AAV9, AAVrh10, AAVretrograde or PHP.eB capsid protein.

5. The AAV capsid protein of any one of claims 1 to 4, wherein the unmodified AAV capsid protein is a wild-type PHP.eB capsid protein, or an AAV capsid protein comprising a sequence having at least 80% sequence identity to SEQ ID NO: 10.

6. The AAV capsid protein of claim 5, wherein the amino acid sequence is inserted between amino acids 588 and 589 of SEQ ID NO: 10, or at an equivalent position in a sequence having at least 80% sequence identity to SEQ ID NO: 10.

7. The AAV capsid protein of any one of claims 1 to 6, wherein:
(a) the AAV capsid protein has the amino acid sequence of SEQ ID NO: 11;
(b) the AAV capsid protein has the amino acid sequence of SEQ ID NO: 12;
(c) the AAV capsid protein has the amino acid sequence of SEQ ID NO: 13;
(d) the AAV capsid protein has the amino acid sequence of SEQ ID NO: 14;
(e) the AAV capsid protein has the amino acid sequence of SEQ ID NO: 15;
(f) the AAV capsid protein has the amino acid sequence of SEQ ID NO: 16;
(g) the AAV capsid protein has the amino acid sequence of SEQ ID NO: 17;
(h) the AAV capsid protein has the amino acid sequence of SEQ ID NO: 18; or
(i) the AAV capsid protein has the amino acid sequence of SEQ ID NO: 19.

8. A nucleic acid encoding the AAV capsid protein of any one of claims 1 to 7.

9. The nucleic acid of claim 8, wherein:
(a) the nucleic acid has the nucleotide sequence of SEQ ID NO: 20;
(b) the nucleic acid has the nucleotide sequence of SEQ ID NO: 21;
(c) the nucleic acid has the nucleotide sequence of SEQ ID NO: 22;
(d) the nucleic acid has the nucleotide sequence of SEQ ID NO: 23;
(e) the nucleic acid has the nucleotide sequence of SEQ ID NO: 24;
(f) the nucleic acid has the nucleotide sequence of SEQ ID NO: 25;
(g) the nucleic acid has the nucleotide sequence of SEQ ID NO: 26;
(h) the nucleic acid has the nucleotide sequence of SEQ ID NO: 27; or
(i) the nucleic acid has the nucleotide sequence of SEQ ID NO: 28.

10. A recombinant DNA comprising the nucleic acid of claim 8 or 9.

11. A host cell comprising the nucleic acid of claim 8 or 9; or the recombinant DNA of claim 10.

12. A viral particle comprising the AAV capsid protein of any one of claims 1 to 7.

13. The viral particle of claim 12, wherein the viral particle further comprises a recombinant polynucleotide that encodes:
(a) a gene of interest;
(b) a gene editing construct;
(c) an antibody or antigen-binding fragment; or
(d) a gene silencing construct.

14. The viral particle of claim 13, wherein:
(a) the gene of interest is TREM2, CCL4/CCL3, CD22 or CSF1R;
(b) the gene editing construct targets TREM2, CCL4/CCL3, CD22 or CSF1R;
(c) the antibody or antigen-binding fragment binds to TREM2, CCL4/CCL3, CD22 or CSF1R; or
(d) the gene silencing construct downregulates the expression of TREM2, CCL4/CCL3, CD22 or CSF1R.

15. The viral particle of claims 13 or 14, wherein the recombinant polynucleotide encoding the gene of interest, the gene editing construct, the antibody or antigen-binding fragment, or the gene silencing construct further comprises a microglia-specific promoter or enhancer, or a macrophage-specific promoter or enhancer.

16. The viral particle of claim 15, wherein the microglia-specific promoter or enhancer is derived from:
(a) TMEM119;
(b) CX3CR1; or
(c) P2Y12 (P2RY12).

17. The viral particle of claim 15, wherein the macrophage-specific promoter or enhancer is derived from:
(a) CD11b;
(b) CD68;
(c) CSF1R; or
(d) F4/80.

18. A host cell that produces the viral particle of any one of claims 12 to 17.

19. A non-human transgenic animal comprising the viral particle of any one of claims 12 to 17.

20. A pharmaceutical composition comprising the AAV capsid protein of any one of claims 1 to 7; the nucleic acid of claim 8 or 9; or the viral particle of any one of claims 12 to 17, and one or more pharmaceutically acceptable excipients.

21. The capsid protein of any one of claims 1 to 6; the nucleic acid of claim 7 or 8; or the viral particle of any one of claims 11 to 15 for use in therapy and/or diagnostics.

22. A method of targeting microglia or brain macrophages using an AAV capsid protein of the invention, the method comprising introducing a recombinant AAV vector into a mammal, the recombinant AAV vector encoding for a gene of interest, a gene editing construct, an antibody or antigen-binding fragment, or a gene silencing construct, which is encapsidated into a capsid protein of any one of claims 1 to 7.


SEQUENCE LISTING (365 sequences)

SEQ ID NO: 1 - protein, 7 aa, Artificial Sequence
  Amino acid sequence of insertion sequence 1
YAFGGEG

SEQ ID NO: 2 - protein, 7 aa, Artificial Sequence
  Amino acid sequence of insertion sequence 2
ALAVPFR

SEQ ID NO: 3 - protein, 7 aa, Artificial Sequence
  Amino acid sequence of insertion sequence 3
HGTAASH

SEQ ID NO: 4 - protein, 7 aa, Artificial Sequence
  Amino acid sequence of insertion sequence 4
IFVMLVR

SEQ ID NO: 5 - protein, 7 aa, Artificial Sequence
  Amino acid sequence of insertion sequence 5
LHQIPDS

SEQ ID NO: 6 - protein, 7 aa, Artificial Sequence
  Amino acid sequence of insertion sequence 6
LYPLIDL

SEQ ID NO: 7 - protein, 7 aa, Artificial Sequence
  Amino acid sequence of insertion sequence 7
AGTTGFP

SEQ ID NO: 8 - protein, 7 aa, Artificial Sequence
  Amino acid sequence of insertion sequence 8
RLAEITG

SEQ ID NO: 9 - protein, 7 aa, Artificial Sequence
  Amino acid sequence of insertion sequence 9
ALAVPFK

SEQ ID NO: 10 - protein, 736 aa, Artificial Sequence
  PhP.eB
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 743 aa, Artificial Sequence
  Novel capsid protein 1 comprising insertion sequence 1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGYAFGGEGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 743 aa, Artificial Sequence
  Novel capsid protein 2 comprising insertion sequence 2
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGALAVPFRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 743 aa, Artificial Sequence
  Amino acid sequence of novel capsid protein 3 comprising insertion sequence 3
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGHGTAASHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 743 aa, Artificial Sequence
  Amino acid sequence of novel capsid protein 4 comprising insertion sequence 4.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGIFVMLVRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 743 aa, Artificial Sequence
  Amino acid sequence of novel capsid protein 5 comprising insertion sequence 5
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGLHQIPDSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 743 aa, Artificial Sequence
  Amino acid sequence of novel capsid protein 6 comprising insertion sequence 6
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGLYPLIDLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 743 aa, Artificial Sequence
  Amino acid sequence of novel capsid protein 7 comprising insertion sequence 7.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAGTTGFPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 743 aa, Artificial Sequence
  Amino acid sequence of novel capsid protein 8 comprising insertion sequence 8.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGRLAEITGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 743 aa, Artificial Sequence
  Amino acid sequence of novel capsid protein 9 comprising insertion sequence 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGALAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - DNA, 2229 bp, Artificial Sequence
  DNA sequence of novel capsid protein 1 comprising insertion sequence 1
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggtatgcgtttggcggagagggggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 21 - DNA, 2229 bp, Artificial Sequence
  DNA sequence of novel capsid protein 2 comprising insertion sequence 2.
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatggggctttggcggtgcctttcagggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 22 - DNA, 2229 bp, Artificial Sequence
  DNA sequence of novel capsid protein 3 comprising insertion sequence 3.
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggcatgggacagcggcatcgcatgcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 23 - DNA, 2229 bp, Artificial Sequence
  DNA sequence of novel capsid protein 4 comprising insertion sequence 4.
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggatttttgtgatgctcgtcagggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 24 - DNA, 2229 bp, Artificial Sequence
  DNA sequence of novel capsid protein 5 comprising insertion sequence 5
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggttgcatcaaattcctgatagtgcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 25 - DNA, 2229 bp, Artificial Sequence
  DNA sequence of novel capsid protein 6 comprising insertion sequence 6
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggttgtatcctttgattgatctcgcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 26 - DNA, 2229 bp, Artificial Sequence
  DNA sequence of novel capsid protein 7 comprising insertion sequence 7.
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatggggctggcacgacaggtttcccggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 27 - DNA, 2229 bp, Artificial Sequence
  DNA sequence of novel capsid protein 8 comprising insertion sequence 8.
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggcgtttggcggagatcacgggggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 28 - DNA, 2229 bp, Artificial Sequence
  DNA sequence of novel capsid protein 9 comprising insertion sequence 9.
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatggggctttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 29 - DNA, 21 bp, Artificial Sequence
  DNA sequence of insertion sequence 1
tatgcgtttggcggagagggg

SEQ ID NO: 30 - DNA, 21 bp, Artificial Sequence
  DNA sequence of insertion sequence 2
gctttggcggtgcctttcagg

SEQ ID NO: 31 - DNA, 21 bp, Artificial Sequence
  DNA sequence of insertion sequence 3
catgggacagcggcatcgcat

SEQ ID NO: 32 - DNA, 21 bp, Artificial Sequence
  DNA sequence of insertion sequence 4
atttttgtgatgctcgtcagg

SEQ ID NO: 33 - DNA, 21 bp, Artificial Sequence
  DNA sequence of insertion sequence 5
ttgcatcaaattcctgatagt

SEQ ID NO: 34 - DNA, 21 bp, Artificial Sequence
  DNA sequence of insertion sequence 6
ttgtatcctttgattgatctc

SEQ ID NO: 35 - DNA, 21 bp, Artificial Sequence
  DNA sequence of insertion sequence 7
gctggcacgacaggtttcccg

SEQ ID NO: 36 - DNA, 21 bp, Artificial Sequence
  DNA sequence of insertion sequence 8
cgtttggcggagatcacgggg

SEQ ID NO: 37 - DNA, 21 bp, Artificial Sequence
  DNA sequence of insertion sequence 9
gctttggcggtgccttttaag

SEQ ID NO: 38 - DNA, 60 bp, Artificial Sequence
  Forward primer sequence that hybridises to PHP.eB replication centre.
ctaggggttcctgcggcctctagagccaccatgttcaaatttgaactgactaagcggctc

SEQ ID NO: 39 - DNA, 18 bp, Artificial Sequence
  Reverse primer sequence that hybridises to PHP.eB replication centre
agcggtcgcagaggagcg

SEQ ID NO: 40 - DNA, 41 bp, Artificial Sequence
  Forward primer sequence that hybridises to the 3' capsid protein of PHP.eB
tcccgctcctctgcgaccgctatggctgccgatggttatct

SEQ ID NO: 41 - DNA, 25 bp, Artificial Sequence
  Reverse primer sequence that hybridises to the 3' capsid protein of PHP.eB
cagattacgagtcaggtatctggtg

SEQ ID NO: 42 - DNA, 49 bp, Artificial Sequence
  Forward primer sequence that hybridises to T2A-GFP
gatacctgactcgtaatctggagggcagaggaagtctgctaacatgcgg

SEQ ID NO: 43 - DNA, 61 bp, Artificial Sequence
  Reverse primer sequence that hybridises to T2A-GFP
actaggggttcctgcggccgcacacaaaaaaccaacacacagatctaatggctccgggtat

SEQ ID NO: 44 - DNA, 28 bp, Artificial Sequence
  Forward primer sequence that hybridises to PHP.eB backbone
gtcgacttgcttgttaatcaataaaccg

SEQ ID NO: 45 - DNA, 22 bp, Artificial Sequence
  Reverse primer sequence that hybridises to PHP.eB backbone
cccatcactctggtggtttgtg

SEQ ID NO: 46 - DNA, 19 bp, Artificial Sequence
  Forward primer sequence that hybridises to PHP.eB binding arm
ttgatgaatcctggacctg

SEQ ID NO: 47 - DNA, 20 bp, Artificial Sequence
  Reverse primer sequence that hybridises to PHP.eB binding arm
agttcagcttgtccttgttg

SEQ ID NO: 48 - DNA, 100 bp, Artificial Sequence
  DNA sequence of the random library fragment
  misc_feature (41)..(61): n is a, c, g, or t
gtattccttggttttgaacccaaccggtctgcgcctgtgcnnnnnnnnnnnnnnnnnnnnncccatcactctggtggtttgtggccacttgtccatagga

SEQ ID NO: 49 - DNA, 672 bp, Artificial Sequence
  DNA sequence of the silenced PHP.eB capsid protein
atggctgccgatggttgacttccagattaactcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggatgaaagaggcctgtagagtgatctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataactgaggtgccgatggagtgggtagttcc

SEQ ID NO: 50 - DNA, 100 bp, Artificial Sequence
  DNA sequence of the split capsid protein comprising the DNA sequence of insertion sequence 9
tcctatggacaagtggccacaaaccaccagagtgatggggctttggcggtgccttttaaggcacaggcgcagaccggttgggttcaaaaccaaggaatac

SEQ ID NO: 51 - DNA, 100 bp, Artificial Sequence
  DNA sequence of the split capsid protein comprising the DNA sequence of insertion sequence 2
tcctatggacaagtggccacaaaccaccagagtgatggggctttggcggtgcctttcagggcacaggcgcagaccggttgggttcaaaaccaaggaatac

SEQ ID NO: 52 - DNA, 100 bp, Artificial Sequence
  DNA sequence of the split capsid protein comprising the DNA sequence of insertion sequence 3
tcctatggacaagtggccacaaaccaccagagtgatgggcatgggacagcggcatcgcatgcacaggcgcagaccggttgggttcaaaaccaaggaatac

SEQ ID NO: 53 - DNA, 100 bp, Artificial Sequence
  DNA sequence of the split capsid protein comprising the DNA sequence of insertion sequence 1
tcctatggacaagtggccacaaaccaccagagtgatgggtatgcgtttggcggagagggggcacaggcgcagaccggttgggttcaaaaccaaggaatac

SEQ ID NO: 54 - protein, 7 aa, artificial sequence
  Insertion sequence of Table 4
IFFLNVT

SEQ ID NO: 55 - protein, 7 aa, artificial sequence
  Insertion sequence of Table 4
IFFLNDT

SEQ ID NO: 56 - protein, 7 aa, artificial sequence
  Insertion sequence of Table 4
IFFFNVT

SEQ ID NO: 57 - protein, 7 aa, artificial sequence
  Insertion sequence of Table 4
SFFLNYT

SEQ ID NO: 58 - protein, 7 aa, artificial sequence
  Insertion sequence of Table 4
IFFFNDT

SEQ ID NO: 59 - protein, 7 aa, artificial sequence
  Insertion sequence of Table 4
SFFFNDT

SEQ ID NO: 60 - protein, 7 aa, artificial sequence
  Insertion sequence of Table 4
IFFLNYT

SEQ ID NO: 61 - protein, 7 aa, artificial sequence
  Insertion sequence of Table 4
SFFFNYT

SEQ ID NO: 62 - protein, 7 aa, artificial sequence
  Insertion sequence of Table 4
SFFLNDT

SEQ ID NO: 63 - protein, 7 aa, artificial sequence
  Insertion sequence of Table 4
IFFFNYT

SEQ ID NO: 64 - protein, 7 aa, artificial sequence
  Insertion sequence of Table 4
SFFLNVT

SEQ ID NO: 65 - protein, 7 aa, artificial sequence
  Insertion sequence of Table 4
FFFLNVT

SEQ ID NO: 66 - protein, 7 aa, artificial sequence
  Insertion sequence of Table 4
SFFLTVT

SEQ ID NO: 67 - protein, 7 aa, artificial sequence
  Insertion sequence of Table 4
FFFFNYT

SEQ ID NO: 68 - protein, 7 aa, artificial sequence
  Insertion sequence of Table 4
FFFFNDT

SEQ ID NO: 69 - protein, 7 aa, artificial sequence
  Insertion sequence of Table 4
SFFFNVT

SEQ ID NO: 70 - protein, 7 aa, artificial sequence
  Insertion sequence of Table 4
FFFFNVT

SEQ ID NO: 71 - protein, 7 aa, artificial sequence
  Insertion sequence of Table 4
FFVLNVT

SEQ ID NO: 72 - protein, 7 aa, artificial sequence
  Insertion sequence of Table 4
PPPPRPP

SEQ ID NO: 73 - protein, 7 aa, artificial sequence
  Insertion sequence of Table 4
NSNSHSF

SEQ ID NO: 74 - protein, 7 aa, artificial sequence
  Insertion sequence of Table 4
IFLFNYT

SEQ ID NO: 75 - protein, 7 aa, artificial sequence
  Insertion sequence of Table 4
FFFLNDT

SEQ ID NO: 76 - protein, 7 aa, artificial sequence
  Insertion sequence of Table 4
SFVFNIT

SEQ ID NO: 77 - protein, 7 aa, artificial sequence
  Insertion sequence of Table 4
FFFLNYT

SEQ ID NO: 78 - protein, 7 aa, artificial sequence
  Insertion sequence of Table 4
IFFLTIR

SEQ ID NO: 79 - protein, 7 aa, artificial sequence
  Insertion sequence of Table 4
NRNLHSN

SEQ ID NO: 80 - protein, 7 aa, artificial sequence
  Insertion sequence of Table 4
SFLLNYT

SEQ ID NO: 81 - protein, 7 aa, artificial sequence
  Insertion sequence of Table 4
FFFLTDQ

SEQ ID NO: 82 - protein, 7 aa, artificial sequence
  Insertion sequence of Table 4
SFFFNCT

SEQ ID NO: 83 - protein, 7 aa, artificial sequence
  Insertion sequence of Table 4
IFVLNYT

SEQ ID NO: 84 - protein, 7 aa, artificial sequence
  Insertion sequence of Table 4
SFFFTVT

SEQ ID NO: 85 - protein, 7 aa, artificial sequence
  Insertion sequence of Table 4
SFSLNYT

SEQ ID NO: 86 - protein, 7 aa, artificial sequence
  Insertion sequence of Table 4
IFVFNYT

SEQ ID NO: 87 - protein, 7 aa, artificial sequence
  Insertion sequence of Table 4
IFFFTYT

SEQ ID NO: 88 - protein, 7 aa, artificial sequence
  Insertion sequence of Table 4
SFLFNYT

SEQ ID NO: 89 - protein, 7 aa, artificial sequence
  Insertion sequence of Table 4
FFFFTDT

SEQ ID NO: 90 - protein, 7 aa, artificial sequence
  Insertion sequence of Table 4
IFFLTYT

SEQ ID NO: 91 - protein, 7 aa, artificial sequence
  Insertion sequence of Table 4
SFVLTVQ

SEQ ID NO: 92 - protein, 7 aa, artificial sequence
  Insertion sequence of Table 4
SFSFTYT

SEQ ID NO: 93 - protein, 7 aa, artificial sequence
  Insertion sequence of Table 4
NFFLNYT

SEQ ID NO: 94 - protein, 7 aa, artificial sequence
  Insertion sequence of Table 4
SFSLNVT

SEQ ID NO: 95 - protein, 7 aa, artificial sequence
  Insertion sequence of Table 4
FFLFNVT

SEQ ID NO: 96 - protein, 7 aa, artificial sequence
  Insertion sequence of Table 4
FFVLIDT

SEQ ID NO: 97 - protein, 7 aa, artificial sequence
  Insertion sequence of Table 4
IFVLNDT

SEQ ID NO: 98 - protein, 7 aa, artificial sequence
  Insertion sequence of Table 4
AFVLNYT

SEQ ID NO: 99 - protein, 7 aa, artificial sequence
  Insertion sequence of Table 4
FFLLNVT

SEQ ID NO: 100 - protein, 7 aa, artificial sequence
  Insertion sequence of Table 4
SFFLNCT

SEQ ID NO: 101 - protein, 7 aa, artificial sequence
  Insertion sequence of Table 4
IFFFNVP

SEQ ID NO: 102 - protein, 7 aa, artificial sequence
  Insertion sequence of Table 4
LFFLNYT

SEQ ID NO: 103 - protein, 7 aa, artificial sequence
  Insertion sequence of Table 4
FFVKHIT

SEQ ID NO: 104 - protein, 7 aa, artificial sequence
  Insertion sequence of Table 4
IFSKRYR

SEQ ID NO: 105 - protein, 7 aa, artificial sequence
  Insertion sequence of Table 4
FFFLTVT

SEQ ID NO: 106 - protein, 7 aa, artificial sequence
  Insertion sequence of Table 4
IFFFTVT

SEQ ID NO: 107 - protein, 7 aa, artificial sequence
  Insertion sequence of Table 4
SFFFTDT

SEQ ID NO: 108 - protein, 7 aa, artificial sequence
  Insertion sequence of Table 4
FFFLTDT

SEQ ID NO: 109 - protein, 7 aa, artificial sequence
  Insertion sequence of Table 4
IFFLTDT

SEQ ID NO: 110 - protein, 7 aa, artificial sequence
  Insertion sequence of Table 4
AFFLTYT

SEQ ID NO: 111 - protein, 7 aa, artificial sequence
  Insertion sequence of Table 4
NFFLTYP

SEQ ID NO: 112 - protein, 7 aa, artificial sequence
  Insertion sequence of Table 4
IFFFSYT

SEQ ID NO: 113 - protein, 7 aa, artificial sequence
  Insertion sequence of Table 4
IFLFNVT

SEQ ID NO: 114 - protein, 7 aa, artificial sequence
  Insertion sequence of Table 4
PRDLHSN

SEQ ID NO: 115 - protein, 7 aa, artificial sequence
  Insertion sequence of Table 4
SFLLNVT

SEQ ID NO: 116 - protein, 7 aa, artificial sequence
  Insertion sequence of Table 4
HSNSHSF

SEQ ID NO: 117 - protein, 7 aa, artificial sequence
  Insertion sequence of Table 4
HRDLHSN

SEQ ID NO: 118 - protein, 7 aa, artificial sequence
  Insertion sequence of Table 4
NFLLNYT

SEQ ID NO: 119 - protein, 7 aa, artificial sequence
  Insertion sequence of Table 4
IFVFNDT

SEQ ID NO: 120 - protein, 7 aa, artificial sequence
  Insertion sequence of Table 4
IFFLTVT

SEQ ID NO: 121 - protein, 7 aa, artificial sequence
  Insertion sequence of Table 4
FFFLILR

SEQ ID NO: 122 - protein, 7 aa, artificial sequence
  Insertion sequence of Table 4
IWGLLLR

SEQ ID NO: 123 - protein, 7 aa, artificial sequence
  Insertion sequence of Table 4
FFFLTLR

SEQ ID NO: 124 - protein, 7 aa, artificial sequence
  Insertion sequence of Table 4
IFFLMIR

SEQ ID NO: 125 - protein, 7 aa, artificial sequence
  Insertion sequence of Table 4
HMKAHSN

SEQ ID NO: 126 - protein, 7 aa, artificial sequence
  Insertion sequence of Table 4
HRNLHSY

SEQ ID NO: 127 - protein, 7 aa, artificial sequence
  Insertion sequence of Table 4
FFFKDTT

SEQ ID NO: 128 - protein, 7 aa, artificial sequence
  Insertion sequence of Table 4
SFVLNYT

SEQ ID NO: 129 - protein, 7 aa, artificial sequence
  Insertion sequence of Table 4
FFFKNPT

SEQ ID NO: 130 - protein, 7 aa, artificial sequence
  Insertion sequence of Table 4
NYRDLHS

SEQ ID NO: 131 - protein, 7 aa, artificial sequence
  Insertion sequence of Table 4
FFLKHLA

SEQ ID NO: 132 - protein, 7 aa, artificial sequence
  Insertion sequence of Table 4
PWELHSN

SEQ ID NO: 133 - protein, 7 aa, artificial sequence
  Insertion sequence of Table 4
IFFFTDT

SEQ ID NO: 134 - protein, 7 aa, artificial sequence
  Insertion sequence of Table 4
SFVFNVT

SEQ ID NO: 135 - protein, 7 aa, artificial sequence
  Insertion sequence of Table 4
PFFLTYT

SEQ ID NO: 136 - protein, 7 aa, artificial sequence
  Insertion sequence of Table 4
FFVLNDT

SEQ ID NO: 137 - protein, 7 aa, artificial sequence
  Insertion sequence of Table 4
YFFLNDT

SEQ ID NO: 138 - protein, 7 aa, artificial sequence
  Insertion sequence of Table 4
SFLFNDT

SEQ ID NO: 139 - protein, 7 aa, artificial sequence
  Insertion sequence of Table 4
IFFERCG

SEQ ID NO: 140 - protein, 7 aa, artificial sequence
  Insertion sequence of Table 4
NRELHSN

SEQ ID NO: 141 - protein, 7 aa, artificial sequence
  Insertion sequence of Table 4
NFVFHYT

SEQ ID NO: 142 - protein, 7 aa, artificial sequence
  Insertion sequence of Table 4
AFGELHN

SEQ ID NO: 143 - protein, 7 aa, artificial sequence
  Insertion sequence of Table 4
IFFESSR

SEQ ID NO: 144 - protein, 7 aa, artificial sequence
  Insertion sequence of Table 4
PRDLHIN

SEQ ID NO: 145 - protein, 7 aa, artificial sequence
  Insertion sequence of Table 4
IFLLHVT

SEQ ID NO: 146 - protein, 7 aa, artificial sequence
  Insertion sequence of Table 4
NRNSHSI

SEQ ID NO: 147 - protein, 7 aa, artificial sequence
  Insertion sequence of Table 4
HRTAASH

SEQ ID NO: 148 - protein, 7 aa, artificial sequence
  Insertion sequence of Table 4
NGTAASH

SEQ ID NO: 149 - protein, 7 aa, artificial sequence
  Insertion sequence of Table 4
HGTAASQ

SEQ ID NO: 150 - protein, 7 aa, artificial sequence
  Insertion sequence of Table 4
HGAAASH

SEQ ID NO: 151 - protein, 7 aa, artificial sequence
  Insertion sequence of Table 4
HGTAALN

SEQ ID NO: 152 - protein, 7 aa, artificial sequence
  Insertion sequence of Table 4
NGTAASQ

SEQ ID NO: 153 - protein, 7 aa, artificial sequence
  Insertion sequence of Table 4
NGTAASN

SEQ ID NO: 154 - protein, 7 aa, artificial sequence
  Insertion sequence of Table 4
HLTVASQ

SEQ ID NO: 155 - protein, 7 aa, artificial sequence
  Insertion sequence of Table 4
IFVLNVT

SEQ ID NO: 156 - protein, 7 aa, artificial sequence
  Insertion sequence of Table 4
IFVFNVT

SEQ ID NO: 157 - protein, 7 aa, artificial sequence
  Insertion sequence of Table 4
IFLLMLR

SEQ ID NO: 158 - protein, 7 aa, artificial sequence
  Insertion sequence of Table 4
IFVLNVK

SEQ ID NO: 159 - protein, 7 aa, artificial sequence
  Insertion sequence of Table 4
IFVVNVT

SEQ ID NO: 160 - protein, 7 aa, artificial sequence
  Insertion sequence of Table 4
IFFVMIR

SEQ ID NO: 161 - protein, 7 aa, artificial sequence
  Insertion sequence of Table 4
IFVVPSR

SEQ ID NO: 162 - protein, 7 aa, artificial sequence
  Insertion sequence of Table 4
ALAVAFK

SEQ ID NO: 163 - protein, 7 aa, artificial sequence
  Insertion sequence of Table 4
TLAVPFR

SEQ ID NO: 164 - protein, 7 aa, artificial sequence
  Insertion sequence of Table 4
ILAVPFR

SEQ ID NO: 165 - protein, 7 aa, artificial sequence
  Insertion sequence of Table 4
TLAVPFN

SEQ ID NO: 166 - protein, 7 aa, artificial sequence
  Insertion sequence of Table 4
TLAVPFE

SEQ ID NO: 167 - protein, 7 aa, artificial sequence
  Insertion sequence of Table 4
TLAVPFT

SEQ ID NO: 168 - protein, 7 aa, artificial sequence
  Insertion sequence of Table 4
TLAVPFQ

SEQ ID NO: 169 - protein, 7 aa, artificial sequence
  Insertion sequence of Table 4
TLAVPFM

SEQ ID NO: 170 - protein, 7 aa, artificial sequence
  Insertion sequence of Table 4
TLALPFK

SEQ ID NO: 171 - protein, 7 aa, artificial sequence
  Insertion sequence of Table 4
TLAVLFK

SEQ ID NO: 172 - protein, 7 aa, artificial sequence
  Insertion sequence of Table 4
TLAVPSK

SEQ ID NO: 173 - protein, 7 aa, artificial sequence
  Insertion sequence of Table 4
TSAVPFK

SEQ ID NO: 174 - protein, 7 aa, artificial sequence
  Insertion sequence of Table 4
TLTVPFK

SEQ ID NO: 175 - protein, 7 aa, artificial sequence
  Insertion sequence of Table 4
NLAGPFK

SEQ ID NO: 176 - protein, 7 aa, artificial sequence
  Insertion sequence of Table 4
NLAVPFK

SEQ ID NO: 177 - protein, 7 aa, artificial sequence
  Insertion sequence of Table 4
ILAVPFK

SEQ ID NO: 178 - protein, 7 aa, artificial sequence
  Insertion sequence of Table 4
PLAVPFK

SEQ ID NO: 179 - protein, 7 aa, artificial sequence
  Insertion sequence of Table 4
SLAVPFK

SEQ ID NO: 180 - protein, 7 aa, artificial sequence
  Insertion sequence of Table 4
DLAVPFK

SEQ ID NO: 181 - protein, 7 aa, artificial sequence
  Insertion sequence of Table 4
ALAGPFK

SEQ ID NO: 182 - protein, 7 aa, artificial sequence
  Insertion sequence of Table 4
TLAGPFK

SEQ ID NO: 183 - protein, 7 aa, artificial sequence
  Insertion sequence of Table 4
TWAVPFK

SEQ ID NO: 184 - protein, 7 aa, artificial sequence
  Insertion sequence of Table 4
TFAVPFK

SEQ ID NO: 185 - protein, 7 aa, artificial sequence
  Insertion sequence of Table 4
TLAVHFK

SEQ ID NO: 186 - protein, 7 aa, artificial sequence
  Insertion sequence of Table 4
TLAVPLK

SEQ ID NO: 187 - protein, 7 aa, artificial sequence
  Insertion sequence of Table 4
TLAVTFK

SEQ ID NO: 188 - protein, 7 aa, artificial sequence
  Insertion sequence of Table 4
TVAVPFK

SEQ ID NO: 189 - protein, 7 aa, artificial sequence
  Insertion sequence of Table 4
TLVVPFK

SEQ ID NO: 190 - protein, 7 aa, artificial sequence
  Insertion sequence of Table 4
TLAVPCK

SEQ ID NO: 191 - protein, 7 aa, artificial sequence
  Insertion sequence of Table 4
TLEVPFK

SEQ ID NO: 192 - protein, 7 aa, artificial sequence
  Insertion sequence of Table 4
TLAAPFK

SEQ ID NO: 193 - protein, 7 aa, artificial sequence
  Insertion sequence of Table 4
TLSVPFK

SEQ ID NO: 194 - protein, 7 aa, artificial sequence
  Insertion sequence of Table 4
TLAVRFK

SEQ ID NO: 195 - protein, 7 aa, artificial sequence
  Insertion sequence of Table 4
TLGVPFK

SEQ ID NO: 196 - protein, 7 aa, artificial sequence
  Insertion sequence of Table 4
TLAVPVK

SEQ ID NO: 197 - protein, 7 aa, artificial sequence
  Insertion sequence of Table 4
TLAVAFK

SEQ ID NO: 198 - protein, 7 aa, artificial sequence
  Insertion sequence of Table 4
TLAVSFK

SEQ ID NO: 199 - protein, 7 aa, artificial sequence
  Insertion sequence of Table 4
TLAMPFK

SEQ ID NO: 200 - protein, 7 aa, artificial sequence
  Insertion sequence of Table 4
TMAVPFK

SEQ ID NO: 201 - protein, 7 aa, artificial sequence
  Insertion sequence of Table 4
TLAVPYK

SEQ ID NO: 202 - protein, 7 aa, artificial sequence
  Insertion sequence of Table 4
TLAEPFK

SEQ ID NO: 203 - protein, 7 aa, artificial sequence
  Insertion sequence of Table 4
TLAVPIK

SEQ ID NO: 204 - protein, 7 aa, artificial sequence
  Insertion sequence of Table 4
TFAGPFK

SEQ ID NO: 205 - protein, 7 aa, artificial sequence
  Insertion sequence of Table 4
TLAGPLK

SEQ ID NO: 206 - protein, 7 aa, artificial sequence
  Insertion sequence of Table 4
NLGGPFK

SEQ ID NO: 207 - protein, 7 aa, artificial sequence
  Insertion sequence of Table 4
TLAGAFK

SEQ ID NO: 208 - DNA, 21 bp, artificial sequence
  Insertion sequence of Table 4
attttttttttgaatgttacg

SEQ ID NO: 209 - DNA, 21 bp, artificial sequence
  Insertion sequence of Table 4
attttttttttgaatgatacg

SEQ ID NO: 210 - DNA, 21 bp, artificial sequence
  Insertion sequence of Table 4
atttttttttttaatgttacg

SEQ ID NO: 211 - DNA, 21 bp, artificial sequence
  Insertion sequence of Table 4
tctttttttttaaattatacg

SEQ ID NO: 212 - DNA, 21 bp, artificial sequence
  Insertion sequence of Table 4
atttttttttttaatgatacg

SEQ ID NO: 213 - DNA, 21 bp, artificial sequence
  Insertion sequence of Table 4
tcttttttttttaatgatacg

SEQ ID NO: 214 - DNA, 21 bp, artificial sequence
  Insertion sequence of Table 4
attttttttttgaattatacg

SEQ ID NO: 215 - DNA, 21 bp, artificial sequence
  Insertion sequence of Table 4
tcttttttttttaattatacg

SEQ ID NO: 216 - DNA, 21 bp, artificial sequence
  Insertion sequence of Table 4
tctttttttttgaatgatacg

SEQ ID NO: 217 - DNA, 21 bp, artificial sequence
  Insertion sequence of Table 4
atttttttttttaattatacg

SEQ ID NO: 218 - DNA, 21 bp, artificial sequence
  Insertion sequence of Table 4
tctttttttttgaatgttacg

SEQ ID NO: 219 - DNA, 21 bp, artificial sequence
  Insertion sequence of Table 4
tttttttttttgaatgttacg

SEQ ID NO: 220 - DNA, 21 bp, artificial sequence
  Insertion sequence of Table 4
tctttttttttgactgttacg

SEQ ID NO: 221 - DNA, 21 bp, artificial sequence
  Insertion sequence of Table 4
ttttttttttttaattatacg

SEQ ID NO: 222 - DNA, 21 bp, artificial sequence
  Insertion sequence of Table 4
ttttttttttttaatgatacg

SEQ ID NO: 223 - DNA, 21 bp, artificial sequence
  Insertion sequence of Table 4
tcttttttttttaatgttacg

SEQ ID NO: 224 - DNA, 21 bp, artificial sequence
  Insertion sequence of Table 4
ttttttttttttaatgttacg

SEQ ID NO: 225 - DNA, 21 bp, artificial sequence
  Insertion sequence of Table 4
ttttttgttttgaatgttacg

SEQ ID NO: 226 - DNA, 21 bp, artificial sequence
  Insertion sequence of Table 4
cccccccccccgcgacccccg

SEQ ID NO: 227 - DNA, 21 bp, artificial sequence
  Insertion sequence of Table 4
aacagcaattcacactctttc

SEQ ID NO: 228 - DNA, 21 bp, artificial sequence
  Insertion sequence of Table 4
atttttttgtttaattatacg

SEQ ID NO: 229 - DNA, 21 bp, artificial sequence
  Insertion sequence of Table 4
tttttttttttgaatgatacg

SEQ ID NO: 230 - DNA, 21 bp, artificial sequence
  Insertion sequence of Table 4
tcttttgtttttaatattacg

SEQ ID NO: 231 - DNA, 21 bp, artificial sequence
  Insertion sequence of Table 4
tttttttttttgaattatacg

SEQ ID NO: 232 - DNA, 21 bp, artificial sequence
  Insertion sequence of Table 4
attttttttttaacgatccgg

SEQ ID NO: 233 - DNA, 21 bp, artificial sequence
  Insertion sequence of Table 4
aataggaatttacactctaac

SEQ ID NO: 234 - DNA, 21 bp, artificial sequence
  Insertion sequence of Table 4
tcttttttgttgaattatacg

SEQ ID NO: 235 - DNA, 21 bp, artificial sequence
  Insertion sequence of Table 4
tttttttttttaactgatcag

SEQ ID NO: 236 - DNA, 21 bp, artificial sequence
  Insertion sequence of Table 4
tcttttttttttaattgtacg

SEQ ID NO: 237 - DNA, 21 bp, artificial sequence
  Insertion sequence of Table 4
atttttgttttgaattatacg

SEQ ID NO: 238 - DNA, 21 bp, artificial sequence
  Insertion sequence of Table 4
tcttttttttttactgttacg

SEQ ID NO: 239 - DNA, 21 bp, artificial sequence
  Insertion sequence of Table 4
tctttttctttaaattatacg

SEQ ID NO: 240 - DNA, 21 bp, artificial sequence
  Insertion sequence of Table 4
atttttgtttttaattatacg

SEQ ID NO: 241 - DNA, 21 bp, artificial sequence
  Insertion sequence of Table 4
atttttttttttacgtatacg

SEQ ID NO: 242 - DNA, 21 bp, artificial sequence
  Insertion sequence of Table 4
tcttttttgtttaattatacg

SEQ ID NO: 243 - DNA, 21 bp, artificial sequence
  Insertion sequence of Table 4
ttttttttttttactgatacg

SEQ ID NO: 244 - DNA, 21 bp, artificial sequence
  Insertion sequence of Table 4
attttttttttgacttatacg

SEQ ID NO: 245 - DNA, 21 bp, artificial sequence
  Insertion sequence of Table 4
tcttttgttttgactgttcag

SEQ ID NO: 246 - DNA, 21 bp, artificial sequence
  Insertion sequence of Table 4
tctttttcttttacttatacg

SEQ ID NO: 247 - DNA, 21 bp, artificial sequence
  Insertion sequence of Table 4
aatttttttttgaattatacg

SEQ ID NO: 248 - DNA, 21 bp, artificial sequence
  Insertion sequence of Table 4
tctttttctttgaatgttacg

SEQ ID NO: 249 - DNA, 21 bp, artificial sequence
  Insertion sequence of Table 4
ttttttttgtttaatgttacg

SEQ ID NO: 250 - DNA, 21 bp, artificial sequence
  Insertion sequence of Table 4
ttttttgtgttgattgatacg

SEQ ID NO: 251 - DNA, 21 bp, artificial sequence
  Insertion sequence of Table 4
atttttgttttaaatgatacg

SEQ ID NO: 252 - DNA, 21 bp, artificial sequence
  Insertion sequence of Table 4
gcttttgttttgaattatacg

SEQ ID NO: 253 - DNA, 21 bp, artificial sequence
  Insertion sequence of Table 4
ttttttttgttaaatgttacg

SEQ ID NO: 254 - DNA, 21 bp, artificial sequence
  Insertion sequence of Table 4
tctttttttttaaattgtacg

SEQ ID NO: 255 - DNA, 21 bp, artificial sequence
  Insertion sequence of Table 4
atttttttttttaatgttccg

SEQ ID NO: 256 - DNA, 21 bp, artificial sequence
  Insertion sequence of Table 4
cttttttttttaaattatacg

SEQ ID NO: 257 - DNA, 21 bp, artificial sequence
  Insertion sequence of Table 4
ttttttgtgaagcatattacg

SEQ ID NO: 258 - DNA, 21 bp, artificial sequence
  Insertion sequence of Table 4
attttttctaaacgatacagg

SEQ ID NO: 259 - DNA, 21 bp, artificial sequence
  Insertion sequence of Table 4
tttttttttttaactgttacg

SEQ ID NO: 260 - DNA, 21 bp, artificial sequence
  Insertion sequence of Table 4
atttttttttttactgttacg

SEQ ID NO: 261 - DNA, 21 bp, artificial sequence
  Insertion sequence of Table 4
tcttttttttttacggatacg

SEQ ID NO: 262 - DNA, 21 bp, artificial sequence
  Insertion sequence of Table 4
tttttttttttgactgatacg

SEQ ID NO: 263 - DNA, 21 bp, artificial sequence
  Insertion sequence of Table 4
attttttttttaactgatacg

SEQ ID NO: 264 - DNA, 21 bp, artificial sequence
  Insertion sequence of Table 4
gctttttttttgacttatacg

SEQ ID NO: 265 - DNA, 21 bp, artificial sequence
  Insertion sequence of Table 4
aatttttttttgacttatccg

SEQ ID NO: 266 - DNA, 21 bp, artificial sequence
  Insertion sequence of Table 4
atttttttttttagttatacg

SEQ ID NO: 267 - DNA, 21 bp, artificial sequence
  Insertion sequence of Table 4
atttttttgtttaatgttacg

SEQ ID NO: 268 - DNA, 21 bp, artificial sequence
  Insertion sequence of Table 4
ccccgagatctacacagcaat

SEQ ID NO: 269 - DNA, 21 bp, artificial sequence
  Insertion sequence of Table 4
tcttttttgttgaatgttacg

SEQ ID NO: 270 - DNA, 21 bp, artificial sequence
  Insertion sequence of Table 4
cacagcaattcacactctttc

SEQ ID NO: 271 - DNA, 21 bp, artificial sequence
  Insertion sequence of Table 4
caccgagatctacacagcaat

SEQ ID NO: 272 - DNA, 21 bp, artificial sequence
  Insertion sequence of Table 4
aattttttgttgaattatacg

SEQ ID NO: 273 - DNA, 21 bp, artificial sequence
  Insertion sequence of Table 4
atttttgtttttaatgatacg

SEQ ID NO: 274 - DNA, 21 bp, artificial sequence
  Insertion sequence of Table 4
attttttttttgactgttacg

SEQ ID NO: 275 - DNA, 21 bp, artificial sequence
  Insertion sequence of Table 4
tttttttttttaattttacgg

SEQ ID NO: 276 - DNA, 21 bp, artificial sequence
  Insertion sequence of Table 4
atttgggggttgcttttaagg

SEQ ID NO: 277 - DNA, 21 bp, artificial sequence
  Insertion sequence of Table 4
tttttttttttaactttaagg

SEQ ID NO: 278 - DNA, 21 bp, artificial sequence
  Insertion sequence of Table 4
attttttttttaatgatccgg

SEQ ID NO: 279 - DNA, 21 bp, artificial sequence
  Insertion sequence of Table 4
cacatgaaggcgcactctaac

SEQ ID NO: 280 - DNA, 21 bp, artificial sequence
  Insertion sequence of Table 4
cacaggaatttacactcttac

SEQ ID NO: 281 - DNA, 21 bp, artificial sequence
  Insertion sequence of Table 4
tttttttttaaggatacgacg

SEQ ID NO: 282 - DNA, 21 bp, artificial sequence
  Insertion sequence of Table 4
tcttttgttttgaattatacg

SEQ ID NO: 283 - DNA, 21 bp, artificial sequence
  Insertion sequence of Table 4
tttttttttaagaatccgacg

SEQ ID NO: 284 - DNA, 21 bp, artificial sequence
  Insertion sequence of Table 4
aactaccgagatctacacagc

SEQ ID NO: 285 - DNA, 21 bp, artificial sequence
  Insertion sequence of Table 4
ttttttttgaagcatctggcg

SEQ ID NO: 286 - DNA, 21 bp, artificial sequence
  Insertion sequence of Table 4
ccctgggagctgcacagcaat

SEQ ID NO: 287 - DNA, 21 bp, artificial sequence
  Insertion sequence of Table 4
atttttttttttactgatacg

SEQ ID NO: 288 - DNA, 21 bp, artificial sequence
  Insertion sequence of Table 4
tcttttgtttttaatgttacg

SEQ ID NO: 289 - DNA, 21 bp, artificial sequence
  Insertion sequence of Table 4
cctttttttttaacttatacg

SEQ ID NO: 290 - DNA, 21 bp, artificial sequence
  Insertion sequence of Table 4
ttttttgttttgaatgatacg

SEQ ID NO: 291 - DNA, 21 bp, artificial sequence
  Insertion sequence of Table 4
tatttttttttgaatgatacg

SEQ ID NO: 292 - DNA, 21 bp, artificial sequence
  Insertion sequence of Table 4
tcttttttgtttaatgatacg

SEQ ID NO: 293 - DNA, 21 bp, artificial sequence
  Insertion sequence of Table 4
attttttttgagagatgcggg

SEQ ID NO: 294 - DNA, 21 bp, artificial sequence
  Insertion sequence of Table 4
aaccgagagctacacagcaat

SEQ ID NO: 295 - DNA, 21 bp, artificial sequence
  Insertion sequence of Table 4
aattttgtgtttcattatacg

SEQ ID NO: 296 - DNA, 21 bp, artificial sequence
  Insertion sequence of Table 4
gccttcggggagctacacaac

SEQ ID NO: 297 - DNA, 21 bp, artificial sequence
  Insertion sequence of Table 4
attttttttgagtcatctagg

SEQ ID NO: 298 - DNA, 21 bp, artificial sequence
  Insertion sequence of Table 4
ccccgagatctacacatcaat

SEQ ID NO: 299 - DNA, 21 bp, artificial sequence
  Insertion sequence of Table 4
atttttttgttgcatgttacg

SEQ ID NO: 300 - DNA, 21 bp, artificial sequence
  Insertion sequence of Table 4
aacaggaattcacactctatc

SEQ ID NO: 301 - DNA, 21 bp, artificial sequence
  Insertion sequence of Table 4
catcggacagcggcatcgcat

SEQ ID NO: 302 - DNA, 21 bp, artificial sequence
  Insertion sequence of Table 4
aatgggacagcggcatcgcat

SEQ ID NO: 303 - DNA, 21 bp, artificial sequence
  Insertion sequence of Table 4
catgggacagcggcatcgcag

SEQ ID NO: 304 - DNA, 21 bp, artificial sequence
  Insertion sequence of Table 4
catggggcagcggcatcgcat

SEQ ID NO: 305 - DNA, 21 bp, artificial sequence
  Insertion sequence of Table 4
catgggacagcggcattgaat

SEQ ID NO: 306 - DNA, 21 bp, artificial sequence
  Insertion sequence of Table 4
aatgggacagcggcatcgcag

SEQ ID NO: 307 - DNA, 21 bp, artificial sequence
  Insertion sequence of Table 4
aatgggacagcggcatcgaat

SEQ ID NO: 308 - DNA, 21 bp, artificial sequence
  Insertion sequence of Table 4
catttgacagtggcatctcag

SEQ ID NO: 309 - DNA, 21 bp, artificial sequence
  Insertion sequence of Table 4
atttttgttttgaatgttacg

SEQ ID NO: 310 - DNA, 21 bp, artificial sequence
  Insertion sequence of Table 4
atttttgtttttaatgttacg

SEQ ID NO: 311 - DNA, 21 bp, artificial sequence
  Insertion sequence of Table 4
atttttttgttaatgttacgg

SEQ ID NO: 312 - DNA, 21 bp, artificial sequence
  Insertion sequence of Table 4
atttttgttttgaatgttaag

SEQ ID NO: 313 - DNA, 21 bp, artificial sequence
  Insertion sequence of Table 4
atttttgttgtgaatgttacg

SEQ ID NO: 314 - DNA, 21 bp, artificial sequence
  Insertion sequence of Table 4
attttttttgtaatgatacgg

SEQ ID NO: 315 - DNA, 21 bp, artificial sequence
  Insertion sequence of Table 4
atttttgtggtgccttctagg

SEQ ID NO: 316 - DNA, 21 bp, artificial sequence
  Insertion sequence of Table 4
gctttggcggtggcttttaag

SEQ ID NO: 317 - DNA, 21 bp, artificial sequence
  Insertion sequence of Table 4
actttggcggtgccttttagg

SEQ ID NO: 318 - DNA, 21 bp, artificial sequence
  Insertion sequence of Table 4
attttggcggtgccttttagg

SEQ ID NO: 319 - DNA, 21 bp, artificial sequence
  Insertion sequence of Table 4
actttggcggtgccttttaat

SEQ ID NO: 320 - DNA, 21 bp, artificial sequence
  Insertion sequence of Table 4
actttggcggtgccttttgag

SEQ ID NO: 321 - DNA, 21 bp, artificial sequence
  Insertion sequence of Table 4
actttggcggtgccttttacg

SEQ ID NO: 322 - DNA, 21 bp, artificial sequence
  Insertion sequence of Table 4
actttggcggtgccttttcag

SEQ ID NO: 323 - DNA, 21 bp, artificial sequence
  Insertion sequence of Table 4
actttggcggtgccttttatg

SEQ ID NO: 324 - DNA, 21 bp, artificial sequence
  Insertion sequence of Table 4
actttggcgttgccttttaag

SEQ ID NO: 325 - DNA, 21 bp, artificial sequence
  Insertion sequence of Table 4
actttggcggtgctttttaag

SEQ ID NO: 326 - DNA, 21 bp, artificial sequence
  Insertion sequence of Table 4
actttggcggtgccttctaag

SEQ ID NO: 327 - DNA, 21 bp, artificial sequence
  Insertion sequence of Table 4
acttcggcggtgccttttaag

SEQ ID NO: 328 - DNA, 21 bp, artificial sequence
  Insertion sequence of Table 4
actttgacggtgccttttaag

SEQ ID NO: 329 - DNA, 21 bp, artificial sequence
  Insertion sequence of Table 4
aacttggcgggtccctttaag

SEQ ID NO: 330 - DNA, 21 bp, artificial sequence
  Insertion sequence of Table 4
aacttggcggtgccttttaag

SEQ ID NO: 331 - DNA, 21 bp, artificial sequence
  Insertion sequence of Table 4
attttggcggtgccttttaag

SEQ ID NO: 332 - DNA, 21 bp, artificial sequence
  Insertion sequence of Table 4
cctttggcggtgccttttaag

SEQ ID NO: 333 - DNA, 21 bp, artificial sequence
  Insertion sequence of Table 4
tctttggcggtgccttttaag

SEQ ID NO: 334 - DNA, 21 bp, artificial sequence
  Insertion sequence of Table 4
gatttggcggtgccttttaag

SEQ ID NO: 335 - DNA, 21 bp, artificial sequence
  Insertion sequence of Table 4
gctttggcggggccttttaag

SEQ ID NO: 336 - DNA, 21 bp, artificial sequence
  Insertion sequence of Table 4
actttggcggggccttttaag

SEQ ID NO: 337 - DNA, 21 bp, artificial sequence
  Insertion sequence of Table 4
acttgggcggtgccttttaag

SEQ ID NO: 338 - DNA, 21 bp, artificial sequence
  Insertion sequence of Table 4
acttttgcggtgccttttaag

SEQ ID NO: 339 - DNA, 21 bp, artificial sequence
  Insertion sequence of Table 4
actttggcggtgcattttaag

SEQ ID NO: 340 - DNA, 21 bp, artificial sequence
  Insertion sequence of Table 4
actttggcggtgcctttaaag

SEQ ID NO: 341 - DNA, 21 bp, artificial sequence
  Insertion sequence of Table 4
actttggcggtgacttttaag

SEQ ID NO: 342 - DNA, 21 bp, artificial sequence
  Insertion sequence of Table 4
actgtggcggtgccttttaag

SEQ ID NO: 343 - DNA, 21 bp, artificial sequence
  Insertion sequence of Table 4
actttggtggtgccttttaag

SEQ ID NO: 344 - DNA, 21 bp, artificial sequence
  Insertion sequence of Table 4
actttggcggtgccttgtaag

SEQ ID NO: 345 - DNA, 21 bp, artificial sequence
  Insertion sequence of Table 4
actttggaggtgccttttaag

SEQ ID NO: 346 - DNA, 21 bp, artificial sequence
  Insertion sequence of Table 4
actttggcggcgccttttaag

SEQ ID NO: 347 - DNA, 21 bp, artificial sequence
  Insertion sequence of Table 4
actttgtcggtgccttttaag

SEQ ID NO: 348 - DNA, 21 bp, artificial sequence
  Insertion sequence of Table 4
actttggcggtgcgttttaag

SEQ ID NO: 349 - DNA, 21 bp, artificial sequence
  Insertion sequence of Table 4
actttgggggtgccttttaag

SEQ ID NO: 350 - DNA, 21 bp, artificial sequence
  Insertion sequence of Table 4
actttggcggtgcctgttaag

SEQ ID NO: 351 - DNA, 21 bp, artificial sequence
  Insertion sequence of Table 4
actttggcggtggcttttaag

SEQ ID NO: 352 - DNA, 21 bp, artificial sequence
  Insertion sequence of Table 4
actttggcggtgtcttttaag

SEQ ID NO: 353 - DNA, 21 bp, artificial sequence
  Insertion sequence of Table 4
actttggcgatgccttttaag

SEQ ID NO: 354 - DNA, 21 bp, artificial sequence
  Insertion sequence of Table 4
actatggcggtgccttttaag

SEQ ID NO: 355 - DNA, 21 bp, artificial sequence
  Insertion sequence of Table 4
actttggcggtgccttataag

SEQ ID NO: 356 - DNA, 21 bp, artificial sequence
  Insertion sequence of Table 4
actttggcggagccttttaag

SEQ ID NO: 357 - DNA, 21 bp, artificial sequence
  Insertion sequence of Table 4
actttggcggtgcctattaag

SEQ ID NO: 358 - DNA, 21 bp, artificial sequence
  Insertion sequence of Table 4
acttttgcggggccttttaag

SEQ ID NO: 359 - DNA, 21 bp, artificial sequence
  Insertion sequence of Table 4
actttggcggggcctttaaag

SEQ ID NO: 360 - DNA, 21 bp, artificial sequence
  Insertion sequence of Table 4
aacttgggggggccttttaag

SEQ ID NO: 361 - DNA, 21 bp, artificial sequence
  Insertion sequence of Table 4
actttggccggggcctttaag

SEQ ID NO: 362 - protein, 736 aa, artificial sequence
  AAV6 capsid protein
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 363 - protein, 735 aa, artificial sequence [sequence omitted]

SEQ ID NO: 364 - protein, 743 aa, artificial sequence
  Modified AAV6 capsid protein
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSHGTAASHSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 365 - protein, 742 aa, artificial sequence
  Modified AAV2 capsid protein
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGHGTAASHNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL